(19) United States
(12) Patent Application Publication
(10) Pub. No.: US 2025/0206762 A1
(43) Pub. Date: Jun. 26, 2025

(54) CONFORMATIONALLY GATED OPTOELECTRONIC MOLECULAR RECTIFIERS

(71) Applicant: THE BOARD OF TRUSTEES OF THE UNIVERSITY OF ILLINOIS, Urbana, IL (US)

(72) Inventors: Lisa OLSHANSKY, Champaign, IL (US); Paul GRIFFIN, Urbana, IL (US); Bronte CHARETTE, Champaign, IL (US)

(73) Assignee: THE BOARD OF TRUSTEES OF THE UNIVERSITY OF ILLINOIS, Urbana, IL (US)

(21) Appl. No.: 18/848,429

(22) PCT Filed: Mar. 29, 2023
(86) PCT No.: PCT/US2023/016670

§ 371 Date: Sep. 18, 2024

Related U.S. Application Data
(60) Provisional application No. 63/324,748, filed on Mar. 29, 2022.

(51) Int. Cl. C07F 1/08; C07D 213/36

(52) U.S. Cl. CPC C07F 1/08; C07D 213/36

(57) ABSTRACT

Herein is disclosed a bioinspired approach in which triggered conformational changes are used to control electron transfer (ET) events. Photo-induced conformational rearrangements of a ligand are translated into changes in the coordination geometry and environment about abound metal ion. Taking advantage of the differential coordination properties of Cu^{I} and Cu^{II}, these dynamics facilitate intramolecular ET from Cu^{I} to the ligand to create a CS state. The synthesis and photophysical characterization of CuCl(dpaa^{R}) (dpaa=dipicolylaminoacetophenone, with R═H and OMe) is presented. These ligands incorporate a fluorophore into their framework that gives rise to a twisted intramolecular charge transfer (TICT) excited state. Excited state ligand twisting provides a tetragonal coordination geometry capable of capturing Cu^{II} in the CS state when an internal ortho-OMe binding site is present (as in dpaa^{OMe}).

### RELATED APPLICATIONS

This application claims priority under 35 U.S.C. § 119(e) to U.S. Provisional Patent Application No. 63/324,748, filed Mar. 29, 2022, which is incorporated herein by reference.

### GOVERNMENT SUPPORT

This invention was made with government support under DE-SC0022846 awarded by the Department of Energy. The government has certain rights in the invention.

### BACKGROUND OF THE INVENTION

Malmström, Vallee, and Williams have all proposed that conformational rigidity in blue copper proteins facilitates rapid electron transfer (ET) through the enforcement of an entatic rack-induced state (Biol. Met. 1990, 3, 64; Proc. Natl. Acad. Sci. U.S.A 1968, 59, 498). Cu^{I} is a ‘soft’ d¹⁰ metal ion with no ligand field stabilization and primarily adopts tetrahedral/trigonal geometries, while Cu^{II} is a ‘hard’ d⁹ metal ion and principally adopts square planar or tetragonal coordination geometries. In blue copper proteins, the peptide framework holds the Cu ion in an entatic state: a coordination geometry and environment that is intermediate between those favored by Cu^{I} and Cu^{II}, lowering the reorganization energy associated with ET and hence, speeding it up. An example of these principles comes from the photochemistry of Cu^{I} diimine complexes, Cu(phen)₂ (phen=1,10-phenanthroline). In these systems, metal-to-ligand charge transfer (MLCT) leads to a tetrahedral Cu^{II} excited state that undergoes Jahn-Teller relaxation, flattening to a more tetragonal geometry, which can be trapped in coordinating solvents. This flattening is inhibited for 2,9-functionalized phenanthroline derivatives, where bulky substituents both lock the complex into a tetrahedral coordination geometry and prevent excited state flattening. Counterintuitively, the ³MLCT lifetimes in such systems are elongated. Marcus inverted charge recombination (CR) kinetics, or the preclusion of solvent binding have both been proposed to explain these observations.

Despite the fact that the entatic state principle was initially illustrated in structurally rigid blue copper proteins, entatic state intermediates can also be formed transiently such that their formation is triggered by a conformational change leading to gated ET. In this type of mechanism, enzymes control macromolecular structural changes to preorganize the system such that portions of both inner and outer sphere reorganization energy are paid for in advance.

There is much interest in the construction of a simple molecular electronic device that can act as a molecular rectifier and control the direction of charge flow, for a variety of electronic applications. The disparate time scales of photodriven charge separation (˜fs) and steps in chemical reactions (˜μs) represent an inherent bottleneck to harnessing charge separation processes for chemical reactions. Accordingly, a metal complex that can form a long-lived charge separated state by controlling the direction of charge flow is needed.

### SUMMARY

This disclosure provides ligands that are designed to be dynamic and capable of stabilizing both Cu^{I} and Cu^{II}, in contrast to structurally locked systems. Further, the disclosed ligands comprise twisted intramolecular charge transfer (TICT) fluorophores that undergo triggered conformational rearrangements upon photoexcitation to preferentially stabilize the Cu^{II} state.

Accordingly, this disclosure provides a metal complex of Formula I:

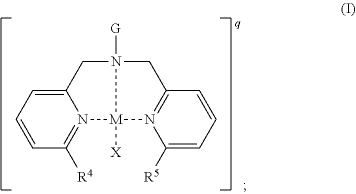

wherein

[symbol] is a dative or a covalent bond;
G is:

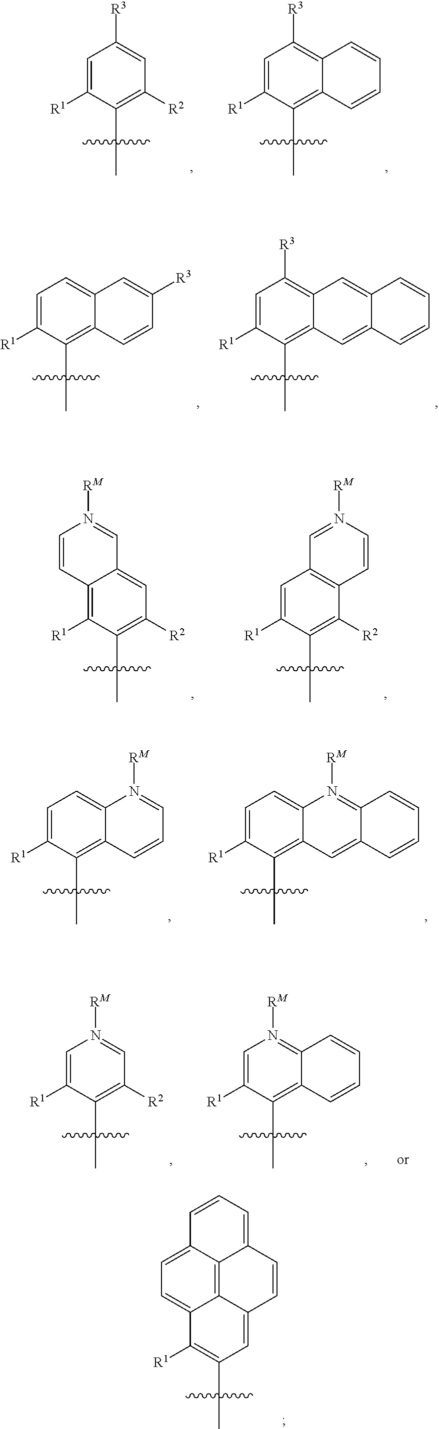

M is a period 4 transition metal or an ion thereof;
R¹ is —O(C₁-C₆)alkyl, —S(C₁-C₆)alkyl, —N[(C₁-C₆)alkyl]₂, —P[(C₁-C₆)alkyl]₂, or —B[(C₁-C₆)alkyl]₂, wherein the heteroatom moiety of R¹ is optionally bonded to M;
R² is H, —O(C₁-C₆)alkyl, —S(C₁-C₆)alkyl, —N[(C₁-C₆)alkyl]₂, —P[(C₁-C₆)alkyl]₂, or —B[(C₁-C₆)alkyl]₂;
R³ is —C(═O)(C₁-C₆)alkyl, —C(═O)OH, —CN, —S(═O)₂(C₁-C₆)alkyl, —S(═O)₂O(C₁-C₆)alkyl, or —C(═O)Ph wherein Ph is optionally substituted;
R⁴ and R are H, or CH₂ bonded to X;
R^{M} is a metal center or complex thereof, O, or a lone pair electrons;
X is halo, or a nitrogen or oxygen heterocycle, when R⁴ and R are H; or
X, when R⁴ and R⁵ are CH₂, is —N(C₁-C₆)alkyl, —P(C₁-C₆)alkyl, —B[(C₁-C₆)alkyl]_{1 or 2}, —B(halo)_{1 or 2}, O, or S; and q is absent or a counter ion.

This disclosure also provides a method for stabilizing charge separation in a transition metal coordination complex comprising:
irradiating a transition metal coordination complex at a suitable excitation wavelength to form a stabilized charge separated state in the complex via a twisted intramolecular charge transfer (TICT) process;
wherein the complex comprises:
   i) a transition metal at a first oxidation state;
   ii) a fluorophore wherein one of its emitting states is comprised of a radical anion localized within the fluorophore;
   iii) a metal coordinating heteroatom covalently bonded to the fluorophore; and
   iv) an electron withdrawing group (EWG) covalently bonded to the fluorophore wherein the EWG is bonded in a position where it is optimally capable of stabilizing the radical anion; and
   v) a multidentate ligand covalently bonded to the fluorophore via a second metal coordinating heteroatom;
wherein the second metal coordinating heteroatom forms a transient radical cation and the fluorophore forms a transient radical anion when the complex is in a TICT state triggered by the irradiation;
the fluorophore has sufficient degrees of freedom to adopt a conformation that allows formation of a dative bond to the transition metal via the metal coordinating heteroatom;
the complex undergoes a conformational rearrangement that stabilizes the charge separated state in the complex; and
the transition metal of the complex in the charge separated state is oxidized to a second oxidation state.

The invention provides novel compounds of Formulas I, II and III, intermediates for the synthesis of compounds of Formulas I, II and III, as well as methods of preparing compounds of Formulas I, II and III. The invention also provides compounds of Formulas I, II and III that are useful as intermediates for the synthesis of other useful compounds.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1A-B. Solid-state structures of CuCl(dpaa^{OMe}) (a) and [CuCl(dpaa^{OMe})]BPh₄ (b) with thermal ellipsoids at 50% plotted in ORTEP. H-atoms, solvent molecules, and counterions are omitted for clarity.

FIG. 2. CVs of 1.0 mM solutions of CuCl(dpaa^{OMe}) (black, outer CV) and [CuCl(dpaa^{OMe})][BPh₄](grey, inner CV) in DMF with 100 mM [NBu₄][PF₆] as supporting electrolyte, scanning at 100 mV/s, and referenced to [Fe(Cp)₂]^{+/0}.

FIG. 3. Absorption (solid lines) and emission spectra (dashed lines) of 0.1 mM solutions of dpaa^{OMe} (lower trace at y-axis) and CuCl(dpaa^{OMe}) (upper trace at y-axis) in DCM (λₑₓ=336 nm).

FIG. 4A-B. Single wavelength TR-PL traces for complexes (lower line) and ligands (upper line) in DCM detected at 485 nm for CuCl(dpaa^{OMe}) and dpaa^{OMe} (a) and 360 nm CuCl(dpaa^{H}) for dpaa^{H}(b). λₑₓ=325 nm in all cases and intensities are normalized to OD₃₂₅ and corrected for data acquisition times. Lines represent fits to Eq. 2.

FIG. 5A-B. fs OTA spectral traces of CuCl(dpaa^{H}) (a), and CuCl(dpaa^{OMe}) (b) in DCM with λₑₓ=340 nm at 492 μW. *Indicates probe noise.

FIG. 6A-B. (a) Proposed Models for the Observed Photophysics of CuCl(dpaa^{OMe}) and CuCl(dpaa^{H}).^{α α}λₘₐₓ of ESAs from OTA spectroscopy are above, and emission maxima from TR-PL spectroscopy are alongside transitions for CuCl(dpaa^{H}) (top number), and CuCl(dpaa^{OMe}) (bottom number). (b) Mechanism of conformationally gated optoelectronic molecular rectifier.

FIG. 7. CVs of Cu^{I} (black) and Cu^{II} (grey) complexes with dpa^{H} (top), and dpa^{OMe} (bottom). Complexes are 1 mM in DMF containing 0.1 M [Bu₄N][PF₆] supporting electrolyte. Data were collected at a glassy carbon working electrode, with a platinum wire counter electrode, and a silver wire pseudo-reference electrode using a scan rate of 100 mV s⁻¹. Open circuit potentials are indicated by arrowhead.

FIG. 8A-B. Thermal ellipsoid plots of the solid state structures of Cu(I) (a) and Cu(II) (b) complexes bound to dpa^{SMe} shown at 50% probability. H-atoms, co-crystalized solvent molecules, and counterions omitted for clarity. Tau values report on the coordination geometries observed, where τ4=0 (square planar), τ4=1 (tetrahedral), τ5=0 (square pyramidal), and τ5=1 (trigonal bipyramidal) are the idealized cases.

FIG. 9A-D. fs Transient absorption (TA) spectra collected upon excitation (Qex=325 nm) of dpan^{OMe} (a), CuCl(dpan^{OMe}) (b), dpan^{H} (c), and CuCl(dpan^{H}) (d) in DMA. Uppermost trace to lowest trace are spectra collected at 1, 5, 10, 25, 50, 100, 200, 1000, and 3000 ps, respectively. Bottom traces represent background signal collected before t=0.

FIG. 10A-B. Thermal ellipsoid plots of the solid state structures of Cu(I) (a) and Cu(II) (b) complexes bound to dpan^{OMe} shown at 50% probability. H-atoms, co-crystalized solvent molecules, and counterions omitted for clarity.

### DETAILED DESCRIPTION

The continued development of solar energy as a renewable resource necessitates the design and study of new approaches to sustaining photodriven charge separation (CS). To this end, we present a bioinspired approach in which triggered conformational changes are used to control electron transfer (ET) events. Herein, photo-induced conformational rearrangements of a ligand are translated into changes in the coordination geometry and environment about a bound metal ion. Taking advantage of the differential coordination properties of Cu^{I} and Cu^{II}, these dynamics facilitate intramolecular ET from Cu^{I} to the ligand to create a CS state. The synthesis and photophysical characterization of CuCl(dpaa^{R}) (dpaa=dipicolylaminoacetophenone, with R═H and OMe) is presented. These ligands incorporate a fluorophore into their framework that gives rise to a twisted intramolecular charge transfer (TICT) excited state. Excited state ligand twisting provides a tetragonal coordination geometry capable of capturing Cu^{II} in the CS state when an internal ortho-OMe binding site is present (as in dpaa^{OMe}). We employ NMR, IR, EPR, and optical spectroscopies, X-ray diffraction, and electrochemical methods to establish the ground state properties of the Cu^{I} and Cu^{II} complexes. We then investigate the photophysical dynamics of these Cu^{I} complexes via time-resolved photoluminescence (TR-PL), and optical transient absorption (OTA) spectroscopies. We show that relative to controls lacking a TICT-active ligand, the lifetime of the CS state is enhanced ˜1000-fold. We also show that the presence of the ortho-OMe substituent on the TICT ligand critically enhances both the yield and lifetime of the CS state, presumably by locking the coordination environment of Cu^{II} in place and imposing a large reorganization energy for charge recombination. These systems represent a first demonstration of our approach to solar energy conversion in which light-driven conformational changes are applied to manipulate the reorganization energy of ET steps.

Additional information and data supporting the invention can be found in the following publications by the inventors: J. Am. Chem. Soc. 2022, 144, 12116-12126 and its Supporting Information, and Dalton Trans., 2022, 51, 6212-6219 and its Supporting Information, which publications are incorporated herein by reference in its entirety.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2, 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

The recitation of a), b), c), . . . or i), ii), iii), or the like in a list of components or steps do not confer any particular order unless explicitly stated.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); for heterocyclic synthesis see Hermanson, Greg T., Bioconjugate Techniques, Third Edition, Academic Press, 2013.

The formulas and compounds described herein can be modified using protecting groups. Suitable amino and carboxy protecting groups are known to those skilled in the art (see for example, Protecting Groups in Organic Synthesis, Second Edition, Greene, T. W., and Wutz, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms; or for example, a range between 1-20 carbon atoms, such as 2-6, 3-6, 2-8, or 3-8 carbon atoms. As used herein, the term “alkyl” also encompasses a “cycloalkyl”, defined below. Examples include, but are not limited to, methyl, ethyl, 1-propyl, 2-propyl (iso-propyl), 1-butyl, 2-methyl-1-propyl (isobutyl), 2-butyl (sec-butyl), 2-methyl-2-propyl (t-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted, for example, with a substituent described below or otherwise described herein. The alkyl can also be optionally partially or fully unsaturated. As such, the recitation of an alkyl group can include an alkenyl group or an alkynyl group.

The alkyl can be a monovalent hydrocarbon radical, as described and exemplified above, or it can be a divalent hydrocarbon radical (i.e., an alkylene).

An alkylene is an alkyl group having two free valences at a carbon atom or two different carbon atoms of a carbon chain. Similarly, alkenylene and alkynylene are respectively an alkene and an alkyne having two free valences at two different carbon atoms, or an alkenylene can have the two free valences on the same carbon.

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like. The cycloalkyl can be unsubstituted or substituted. The cycloalkyl group can be monovalent or divalent, and can be optionally substituted as described for alkyl groups. The cycloalkyl group can optionally include one or more cites of unsaturation, for example, the cycloalkyl group can include one or more carbon-carbon double bonds, such as, for example, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and the like.

The term “heteroatom” refers to any atom in the periodic table that is not carbon or hydrogen. Typically, a heteroatom is O, S, N, P. The heteroatom may also be a halogen, metal or metalloid.

The term “heterocycloalkyl” or “heterocyclyl” refers to a saturated or partially saturated monocyclic, bicyclic, or polycyclic ring containing at least one heteroatom selected from nitrogen, sulfur, oxygen, preferably from 1 to 3 heteroatoms in at least one ring. Each ring is preferably from 3 to 10 membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morpholino, 1,3-diazapane, 1,4-diazapane, 1,4-oxazepane, and 1,4-oxathiapane. The group may be a terminal group or a bridging group.

The term “aryl” refers to an aromatic hydrocarbon group derived from the removal of at least one hydrogen atom from a single carbon atom of a parent aromatic ring system. The radical attachment site can be at a saturated or unsaturated carbon atom of the parent ring system. The aryl group can have from 6 to 30 carbon atoms, for example, about 6-10 carbon atoms. The aryl group can have a single ring (e.g., phenyl) or multiple condensed (fused) rings, wherein at least one ring is aromatic (e.g., naphthyl, dihydrophenanthrenyl, fluorenyl, or anthryl). Typical aryl groups include, but are not limited to, radicals derived from benzene, naphthalene, anthracene, biphenyl, and the like. The aryl can be unsubstituted or optionally substituted with a substituent described below. For example, a phenyl moiety or group may be substituted with one or more substituents Rx where Rx is at the ortho-, meta-, orpara-position, and X is an integer variable of 1 to 5.

The term “heteroaryl” refers to a monocyclic, bicyclic, or tricyclic ring system containing one, two, or three aromatic rings and containing at least one nitrogen, oxygen, or sulfur atom in an aromatic ring. The heteroaryl can be unsubstituted or substituted, for example, with one or more, and in particular one to three, substituents, as described in the definition of “substituted”. Typical heteroaryl groups contain 2-20 carbon atoms in the ring skeleton in addition to the one or more heteroatoms, wherein the ring skeleton comprises a 5-membered ring, a 6-membered ring, two 5-membered rings, two 6-membered rings, or a 5-membered ring fused to a 6-membered ring. Examples of heteroaryl groups include, but are not limited to, 2H-pyrrolyl, 3H-indolyl, 4H-quinolizinyl, acridinyl, benzo[b]thienyl, benzothiazolyl, β-carbolinyl, carbazolyl, chromenyl, cinnolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrrolyl, quinazolinyl, quinolyl, quinoxalinyl, thiadiazolyl, thianthrenyl, thiazolyl, thienyl, triazolyl, tetrazolyl, and xanthenyl. In one embodiment the term “heteroaryl” denotes a monocyclic aromatic ring containing five or six ring atoms containing carbon and 1, 2, 3, or 4 heteroatoms independently selected from non-peroxide oxygen, sulfur, and N(Z) wherein Z is absent or is H, O, alkyl, aryl, or (C₁-C₆)alkylaryl. In some embodiments, heteroaryl denotes an ortho-fused bicyclic heterocycle of about eight to ten ring atoms derived therefrom, particularly a benz-derivative or one derived by fusing a propylene, trimethylene, or tetramethylene diradical thereto.

As used herein, the term “substituted” or “substituent” is intended to indicate that one or more (for example, in various embodiments, 1-10; in other embodiments, 1-6; in some embodiments 1, 2, 3, 4, or 5; in certain embodiments, 1, 2, or 3; and in other embodiments, 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound. Suitable indicated groups include, e.g., alkyl, alkenyl, alkynyl, alkoxy, haloalkyl, hydroxyalkyl, aryl, heteroaryl, heterocyclyl, cycloalkyl, alkanoyl, alkoxycarbonyl, amino, alkylamino, dialkylamino, carboxyalkyl, alkylthio, alkylsulfinyl, and alkylsulfonyl. Substituents of the indicated groups can be those recited in a specific list of substituents described herein, or as one of skill in the art would recognize, can be one or more substituents selected from alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, and cyano. Suitable substituents of indicated groups can be bonded to a substituted carbon atom include F, Cl, Br, I, OR′, OC(O)N(R′)₂, CN, CF₃, OCF₃, R′, 0, S, C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety (e.g., (C₁-C₆)alkyl), and wherein the carbon-based moiety can itself be further substituted. When a substituent is monovalent, such as, for example, F or Cl, it is bonded to the atom it is substituting by a single bond. When a substituent is divalent, such as O, it is bonded to the atom it is substituting by a double bond; for example, a carbon atom substituted with O forms a carbonyl group, C═O.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994.

The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof; such as racemic mixtures, which form part of the present invention. Many organic compounds exist in optically active forms, i.e., they have the ability to rotate the plane of plane-polarized light. In describing an optically active compound, the prefixes D and L, or R and S. are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate (defined below), which may occur where there has been no stereoselection or stereospecificity in a chemical reaction or process.

The terms “racemic mixture” and “racemate” refer to an equimolar mixture of two enantiomeric species, devoid of optical activity.

A “solvent” as described herein can include water or an organic solvent. Examples of organic solvents include hydrocarbons such as toluene, xylene, hexane, and heptane; chlorinated solvents such as methylene chloride, chloroform, and dichloroethane; ethers such as diethyl ether, tetrahydrofuran, and dibutyl ether; ketones such as acetone and 2-butanone; esters such as ethyl acetate and butyl acetate; nitriles such as acetonitrile; alcohols such as methanol, ethanol, and tert-butanol; and aprotic polar solvents such as N,N-dimethylformamide (DMF), N,N-dimethylacetamide (DMA), and dimethyl sulfoxide (DMSO). Solvents may be used alone or two or more of them may be mixed for use to provide a “solvent system”.

The term “molecular rectifier” refers to a single molecule which functions as a one-way conductor of a signal. Described herein are metal complexes capable of long-lived charge separation when triggered by light. The metal complex relies on an energetically favored change in the geometry (or conformation) of its chelated metal, based on its different oxidation states. This change in geometry favors the charge separated state by retarding charge recombination, thereby controlling the direction of the flow of charge. Thus, the metal complexes herein behave as optoelectronic molecular rectifiers.

A bond represented by a series of dashes, such as 2, 3, 4, 5, 6, 7, or 8 dashes and so forth, can represent a covalent bond, dative bond, or a coordinate bond.

### EMBODIMENTS OF THE TECHNOLOGY

1. A metal complex of Formula I:

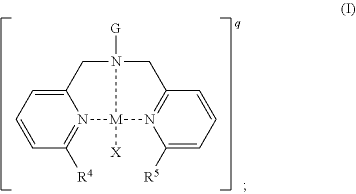

wherein [symbol] is a dative or a covalent bond;
   G is:

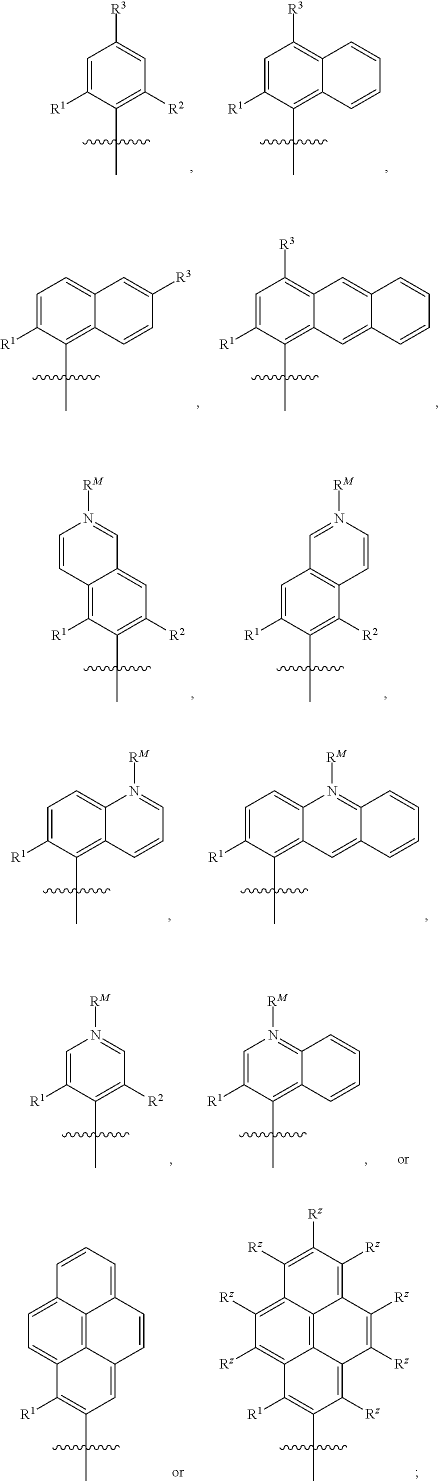

M is a transition metal or an ion thereof;
R¹ is —O(C₁-C₆)alkyl, H, —S(C₁-C₆)alkyl, —N[(C₁-C₆)alkyl]₂, —P[(C₁-C₆)alkyl]₂, or —B[(C₁-C₆)alkyl]₂, wherein the heteroatom moiety of R¹ is optionally bonded to, bonded datively to, or coordinated to M;
R² is H, —O(C₁-C₆)alkyl, —S(C₁-C₆)alkyl, —N[(C₁-C₆)alkyl]₂, —P[(C₁-C₆)alkyl]₂, or —B[(C₁-C₆)alkyl]₂;
R³ is —C(═O)(C₁-C₆)alkyl, H, —C(═O)OH, —C(═O)OR^{A}, —CN, —S(═O)₂(C₁-C₆)alkyl, —S(═O)₂O(C₁-C₆)alkyl, or —C(═O)Ph wherein Ph is optionally substituted;
R^{A} is —(C₁-C₆)alkyl;
R⁴ and R are H, or CH₂ bonded to X;
R^{M} is a metal center or complex thereof, O, or a lone pair electrons;
R^{z} is each independently H, —C(═O)(C₁-C₆)alkyl, H, —C(═O)OH, —C(═O)OR^{A}, —CN, —S(═O)₂(C₁-C₆)alkyl, —S(═O)₂O(C₁-C₆)alkyl, or —C(═O)Ph wherein Ph is optionally substituted;
X is halo, or a nitrogen or oxygen heterocycle or molecule, when R⁴ and R are H; or
X, when R⁴ and R⁵ are CH₂, is —N(C₁-C₆)alkyl, —P(C₁-C₆)alkyl, —B[(C₁-C₆)alkyl]_{1 or 2}, —B(halo)_{1 or 2}, O, or S; and q is absent or a counter ion.

In some embodiments, each R^{Z} is H provided that at least one R is not H at position 1, 4, 5, 6, 7, 8, 9, or 10.

In various embodiments, at least one of R¹, R², and R³ cannot be H. In various embodiments, phenyl is substituted at the ortho-, meta-, or para-position. In various embodiments, M is a transition metal ion capable of undergoing a change in oxidation state such as Cu(I), Cu(II), Ni(0), Ni(II), Pd(0), Pd(II), Pt(0), or Pt(II). In some embodiments, M is Fe(II), Co(II), Mn(I), Mn(II), Mn(III). In some embodiments, R^{M} is a metal center or complex thereof. In some embodiments, the R^{M} comprises an ion of Co, Fe, Os, or Ru. In some embodiments, the ion is a cation or an anion. In some embodiments, the counter ion, q, is BPh₄⁻, PF₆⁻, BF₄⁻, or F₃CSO₃⁻.

In various embodiments, M or R^{M} is a period 4, period 5, period 6, or period 7 transition metal or an ion thereof. In various embodiments, M is Cu(I). In some embodiments, q is absent. In some embodiments, M is Cu(II). In some embodiments, q is a counter ion. In various embodiments, R¹ is —O(C₁-C₆)alkyl. In various embodiments, R³ is —C(═O)OR^{A} wherein R^{A} is —(C₁-C₆)alkyl.

In various embodiments, wherein G is:

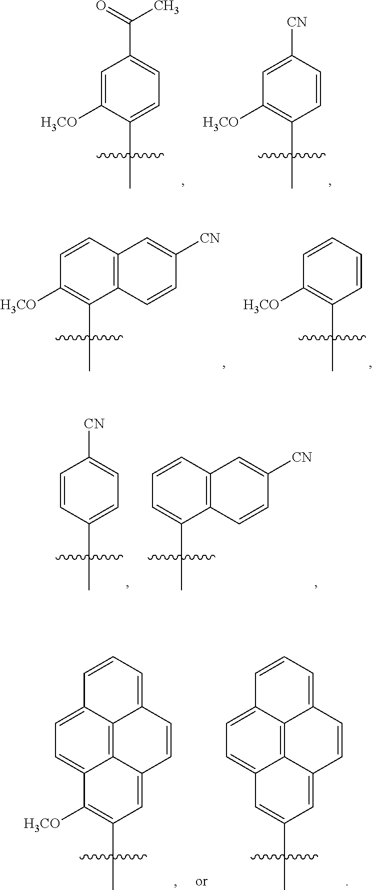

In various embodiments, R³ is —C(═O)(C₁-C₆)alkyl. In various embodiments, R⁴ is H. In various embodiments, R⁵ is H. In various embodiments, X is halo. In various embodiments, X is F, Cl, or I.

In some embodiments, Formula I is represented by Formula II:

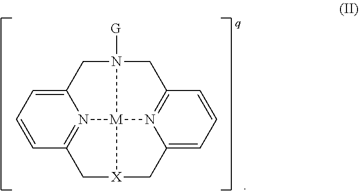

In some embodiments, X is —N(C₁-C₆)alkyl.

In some embodiments, the metal complex is Ia or Ib:

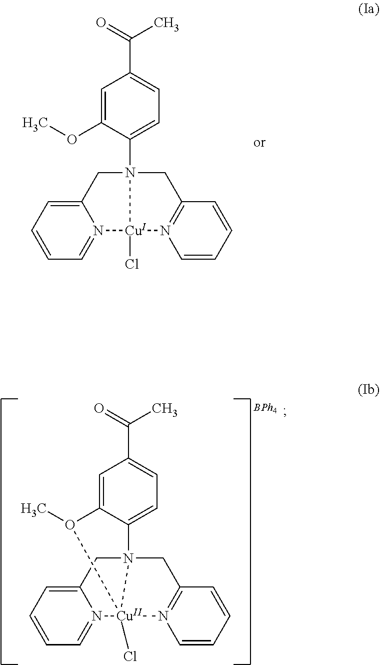

wherein the coordination geometry of Cu(I) of complex Ia is trigonal (such as trigonal planar or tetrahedral), or the coordination geometry of Cu(II) of complex Ib is tetragonal.

This disclosure also provides a semiconductor, a metal organic framework, graphene, a conductive surface, or composition comprising a metal complex described herein.

Also, this disclosure provides a compound of Formula III:

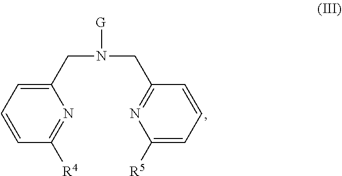

or a salt thereof;
wherein
   G is:

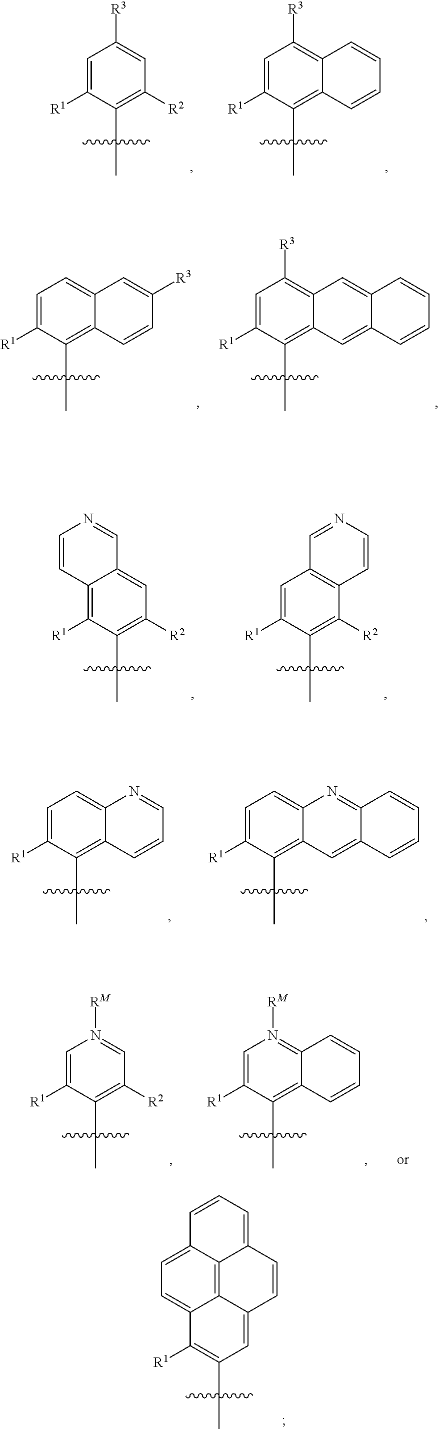

R¹ is —O(C₁-C₆)alkyl, H, —S(C₁-C₆)alkyl, —N[(C₁-C₆)alkyl]₂, —P[(C₁-C₆)alkyl]₂, or —B[(C₁-C₆)alkyl]₂;
R² is H, —O(C₁-C₆)alkyl, —S(C₁-C₆)alkyl, —N[(C₁-C₆)alkyl]₂, —P[(C₁-C₆)alkyl]₂, or —B[(C₁-C₆)alkyl]₂;
R³ is —C(═O)(C₁-C₆)alkyl, H, —C(═O)OH, —CN, —S(═O)₂(C₁-C₆)alkyl, or —S(═O)₂O(C₁-C₆)alkyl, or —C(═O)Ph wherein Ph is optionally substituted;
R⁴ and R are H; or
R⁴ and R taken together is —CH₂—X—CH₂—; and X is —N(C₁-C₆)alkyl, —P(C₁-C₆)alkyl, —B[(C₁-C₆)alkyl]_{1 or 2}, —B(halo)_{1 or 2}, O, or S.

In various embodiments, G is:

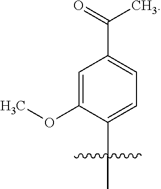 [SMILES: *c1ccc(C(C)=O)cc1OC]

In some embodiments, the compound is IIIa:

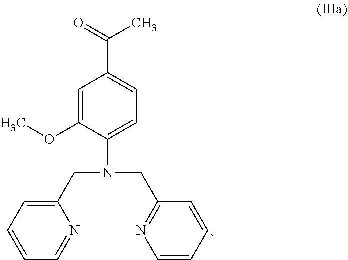 [SMILES: COc1cc(C(C)=O)ccc1N(Cc1ccccn1)Cc1ccccn1]

or a salt thereof.

Additionally, this disclosure provides a composition comprising a compound or complex disclosed herein and another substance. In some embodiments the another substance is a liquid, solvent, solvent, solid, element, mineral, inorganic compound, organic compound, polymer, or copolumer.

Also, this disclosure provides a method for stabilizing charge separation in a metal or transition metal coordination complex comprising:
irradiating a metal or transition metal coordination complex at a suitable excitation wavelength to form a stabilized charge separated state in the complex via a twisted intramolecular charge transfer (TICT) process;
wherein the complex comprises:
   i) a metal or transition metal at a first oxidation state;
   ii) a fluorophore wherein one of its emitting states is comprised of a radical anion localized within the fluorophore;
   iii) a metal or transition metal coordinating heteroatom covalently bonded to the fluorophore; and
   iv) an electron withdrawing group (EWG) covalently bonded to the fluorophore wherein the EWG is bonded in a position where it is optimally capable of stabilizing the radical anion; and
   v) a multidentate ligand covalently bonded to the fluorophore via a second metal or transition metal coordinating heteroatom;
wherein the second metal or transition metal coordinating heteroatom forms a transient radical cation and the fluorophore forms a transient radical anion when the complex is in a TICT state triggered by the irradiation;
the fluorophore has sufficient degrees of freedom to adopt a conformation that allows formation of a dative bond to the transition metal via the metal coordinating heteroatom;
the complex undergoes a conformational rearrangement that stabilizes the charge separated state in the complex; and the metal or transition metal of the complex in the charge separated state is oxidized to a second oxidation state.

In various embodiments, the transition metal coordination complex is represented by Formula I:

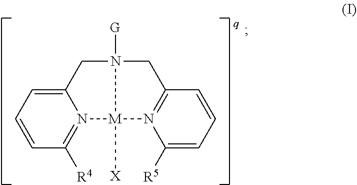

wherein [symbol] is a dative or a covalent bond;
G is:

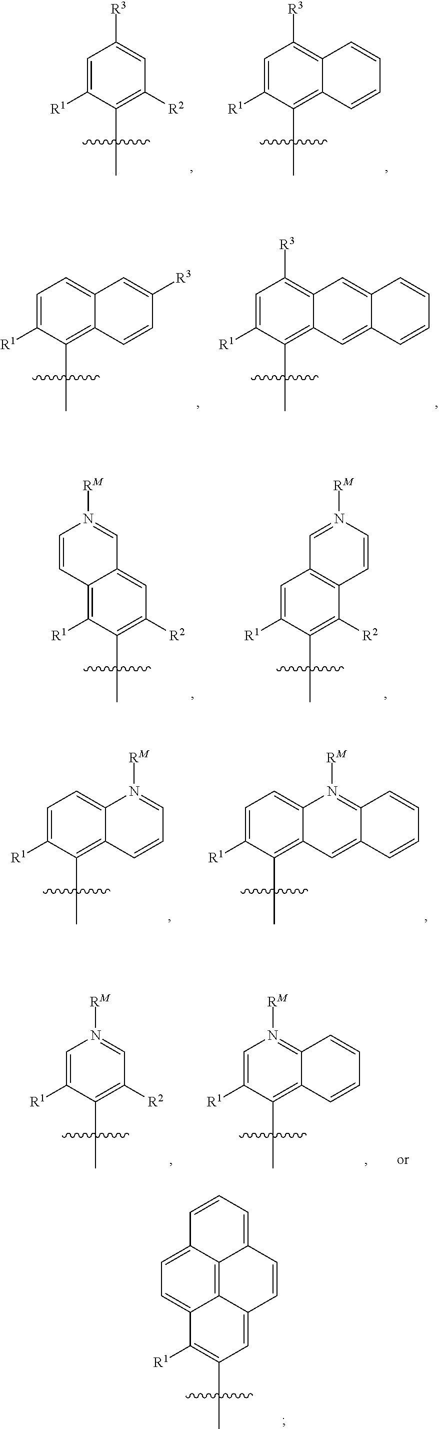

M is a transition metal or an ion thereof;
R¹ is —O(C₁-C₆)alkyl, H, —S(C₁-C₆)alkyl, —N[(C₁-C₆)alkyl]₂, —P[(C₁-C₆)alkyl]₂, or —B[(C₁-C₆)alkyl]₂, wherein the heteroatom moiety of R is optionally bonded to M;
R² is H, —O(C₁-C₆)alkyl, —S(C₁-C₆)alkyl, —N[(C₁-C₆)alkyl]₂, —P[(C₁-C₆)alkyl]₂, or —B[(C₁-C₆)alkyl]₂;
R³ is —C(═O)(C₁-C₆)alkyl, H, —C(═O)OH, —CN, —S(═O)₂(C₁-C₆)alkyl, or —S(═O)₂O(C₁-C₆)alkyl, or —C(═O)Ph wherein Ph is optionally substituted;
R⁴ and R are H or CH₂ bonded to X;
R^{M} is a metal center or complex thereof, O, or a lone pair electrons;
X is halo, or a nitrogen or oxygen heterocycle, when R⁴ and R are H; or
X, when R⁴ and R⁵ are CH₂, is —N(C₁-C₆)alkyl, —P(C₁-C₆)alkyl, —B[(C₁-C₆)alkyl]_{1 or 2}, —B(halo)_{1 or 2}, O, or S; and q is absent or a counter ion.

In some embodiments, the transition metal coordination complex is (Ia):

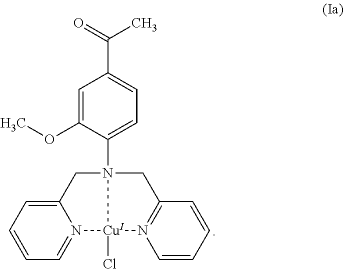

Additionally, this disclosure provides a method for stabilizing charge separation in a transition metal coordination complex comprising irradiating the transition metal coordination complex at a suitable wavelength to induce charge separation in the complex,
wherein the complex comprises:
   i) a transition metal;
   ii) a fluorophore wherein its fluorescence is modulated by the presence or absence of a negative charge;
   iii) a metal coordinating heteroatom covalently bonded to the fluorophore wherein the fluorophore has sufficient degrees of freedom to adopt a conformation that allows formation of a dative bond to the transition metal via the metal coordinating heteroatom when the complex is in an entatic state;
   iv) an electron withdrawing group (EWG) covalently bonded to the fluorophore wherein the EWG is capable of stabilizing a negative charge localized within the fluorophore; and
   v) a multidentate ligand covalently bonded to the fluorophore via a second metal coordinating heteroatom;
wherein the second metal coordinating heteroatom is positively charged and the fluorophore is negatively charged when the complex is in a charge separated state; and the complex changes from tetrahedral conformation in the ground state to a tetragonal conformation in the entatic state thereby stabilizing the charge separation state in the complex.

### Design of Coordination Complex.

As illustrated in Scheme 1, excitation of organic TICT fluorophores promptly generates a locally excited (LE*) state which then undergoes conformational reorganization to afford a twisted, charge-separated TICT* excited state. Both the LE* and TICT* states are emissive but we have designed the ligand fluorophore such that the TICT* state is poised to facilitate ET from bound Cu^{I}. TICT fluorophores consist of an aryl donor-acceptor dyad, typically with a dialkylamine donor and an electron-withdrawing acceptor. In the excited state, twisting of the aryl-N bond generates a radical cation/anion pair. To take advantage of the excited state dynamics of a TICT fluorophore and couple them to the redox dependent conformational changes of Cu^{II/I} coordination complexes, we prepared the ligand shown in Chart 1a. Here, a TICT-active organic fluorophore is appended to a dipicolylamine metal binding site. Further functionalization of the fluorophore is achieved with an ortho-OMe chelating group (Chart 1) to preferentially stabilize the transiently formed Cu^{II} entatic state proposed in Scheme 1.

Chart 1b depicts the series of complexes examined in this work, where the complex in the upper left, CuCl(dpaa^{OMe}), exemplifies our approach to charge separation (CS) and is also featured in Scheme 1. The other complexes in Chart 1b represent control complexes with ligands lacking the TICT axis (dpa^{R}, R=H or OMe), or the ortho-OMe locking group (dpaa^{H}). We have prepared this series of complexes and characterized their ground state properties by single-crystal X-ray diffraction (XRD), ¹H-NMR, IR, EPR, UV-vis absorption and emission spectroscopies, cyclic voltammetry (CV), and mass spectrometry (MS). As we, and others have reported (J. Am. Chem. Soc. 2009, 131, 3230), the Cu^{I} complexes containing an ortho-OMe group exhibit pseudo-tetrahedral coordination geometries in which the ortho-OMe remains unbound to Cu^{I}. In contrast, the Cu^{II} complexes containing an ortho-OMe group are square pyramidal and contain a Cu^{II}—O bond both in solution and in the solid state.

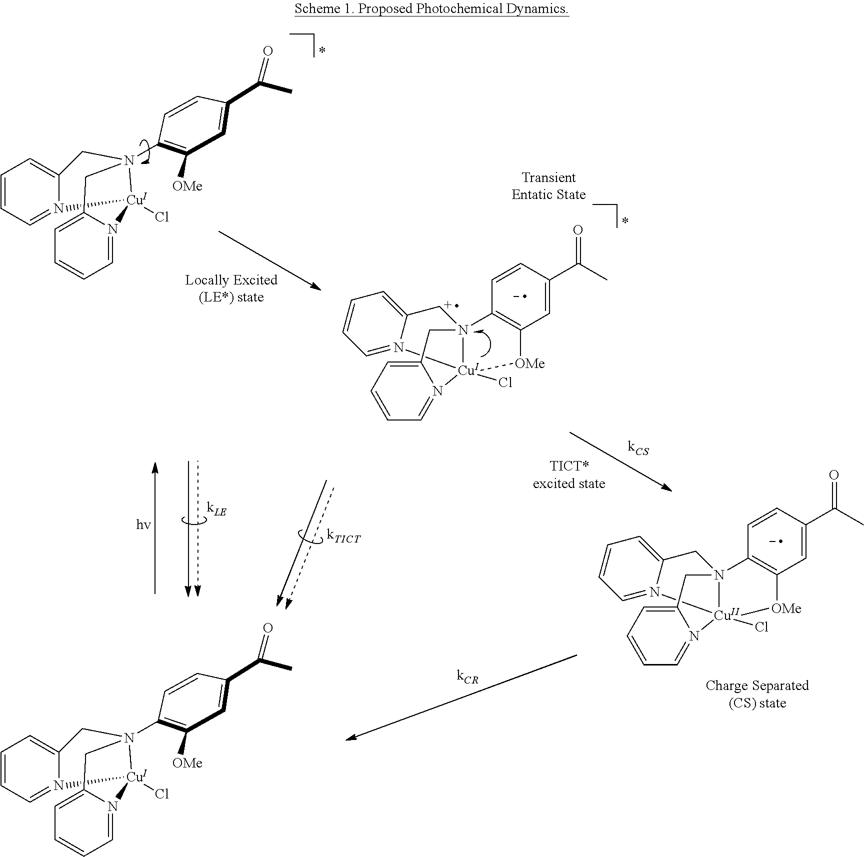

### Chart 1. Ligand Design Principles (a), and Complexes Investigated (b).

(a)

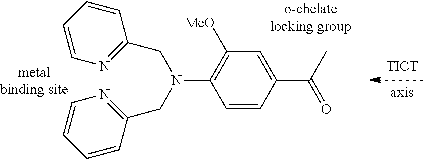

(b)

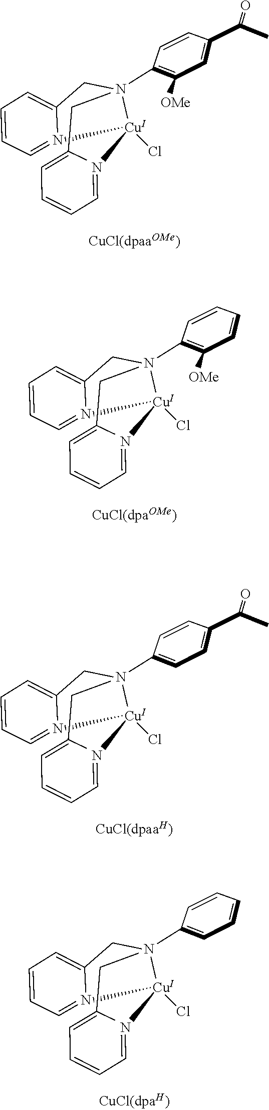

Time-resolved photoluminescence (TR-PL) and optical transient absorption (OTA) spectroscopies were used to investigate the dynamics at play in these systems upon illumination. We found that the TICT axis is critical for the generation of any CS state living longer than 6 ps. Moreover, appreciable CS was only observed in the presence of the ortho-OMe chelate. Specifically, steady state and time-resolved emission measurements reveal a nearly 20-fold increase in emission quenching for CuCl(dpaa^{OMe}) versus with the free ligand (dpaa^{OMe}), while emission from CuCl(dpaa^{H}) is not quenched relative to the free ligand (dpaa^{H}). Additionally, we did not observe a discrete CS state upon photoexcitation of CuCl(dpaa^{H}) via fs OTA, while these experiments with CuCl(dpaa^{OMe}) allowed us to observe the CS state directly and gratifyingly it persists beyond the edge of our observation window (3.5 ns). These findings support that our approach successfully employs photo-induced ligand conformational changes to enhance CS and suggests a new strategy towards increased efficiency in the capture and conversion of solar energy.

### Results.

Synthesis of Ligands and Complexes. The ligands dpaa^{OMe} and dpaa^{H} were synthesized by a four-step protection/deprotection approach (Scheme 2 and Scheme 3) in 47 and 31% overall yields, respectively. The acetal protecting group of 1H was labile and required telescoping of the reaction material through all four steps. We initially prepared [Cu(dpaa^{OMe})(MeCN)][B(C₆F₅)₄] according to a literature procedure (J. Am. Chem. Soc. 2009, 131, 3230; Inorg. Chem. 2002, 41, 2209) but found that these acetonitrile adducts undergo reductive dechlorination in DCM. However, synthesis using CuCl provided an inner sphere chloride ligand that enabled isolation of stable CuCl(dpaa^{OMe}) and CuCl(dpaa^{H}) complexes (Chart 1b). Similar to the CuCl(dpa^{R}) complexes reported by Grabowski, et al. (Chem. Rev. 2003, 103, 3899), elemental analysis revealed that in the solid state, the CuCl(dpaa^{OMe}) complex can exist as a μ-Cl bridging dimer with the formula {[Cu(dpaa^{OMe})]₂(μ-Cl) ICuCl₂}.

Single CrystalXRD. To assess how the coordination geometry at Cu changes with oxidation state, single crystals of Cu^{I} and Cu^{II} in complex with dpaa^{OMe} anddpaa^{H} were prepared and subjected to XRD analysis. Crystal and structural refinement data for these four complexes, and for the free dpaa^{OMe} ligand were obtained for analysis.

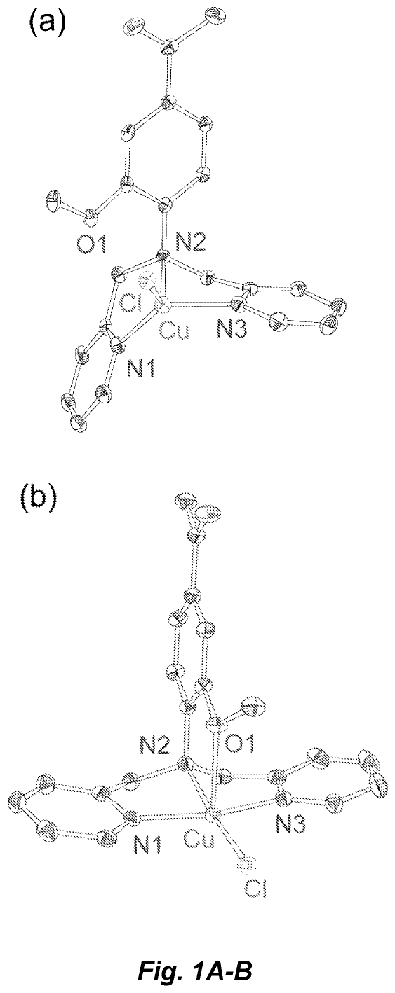

Layer diffusion of pentane into DCM at −36° C. afforded yellow crystals of CuCl(dpaa^{OMe}) and turquoise crystals of [CuCl(dpaa^{OMe})]BPh₄. FIG. 1 provides the solid-state structures of CuCl(dpaa^{OMe}) and [CuCl(dpaa^{OMe})]BPh₄ and select bond lengths for these structures may be found in Table 1. CuCl(dpaa^{OMe}) achieves a tetracoordinate geometry through coordination of the three nitrogen atoms of dpaa^{OMe} and an exogenous Cl⁻ ligand (FIG. 1a). The complex has τ₄ structural parameter of 0.71 indicating that the coordination geometry at Cu^{I} is best described as distorted tetrahedral. The Cu-N_{pyr} bond distances of this complex, Cu—N1 and Cu—N2, of 2.053(1) Å and 2.012(1) Å are unremarkable in the context of Cu^{I} dipicolyamine complexes and consistent with strong bonding interactions (Table 1). The Cu—Nₐₙᵢₗᵢₙₑ distance Cu—N2 of 2.478(1) Å is longer than in the corresponding dipicolylanisidine (2.286(3) Å) and dipicolylthioanisidine (2.181(2) Å) complexes reported by Karlin et al. This lengthening likely results from the comparatively electron-deficient nature of the aniline donor containing acyl substitution in the para-position. Notably, CuCl(dpaa^{OMe}) crystallizes with the methoxy substituent oriented towards Cu, but at a distance of 2.976 Å, indicating it does not bind to Cu^{I} in the solid-state.

TABLE 1
Select Bond Lengths and Distortion Parameters for
CuCl(dpaa^{OMe}) and [CuCl(dpaa^{OMe})][BPh₄].

| | CuCl(dpaa^{OMe}) | [CuCl(dpaa^{OMe})]BPh₄ |
|---|---|---|
| Cu—N₁ | 2.053(1) | 1.994(2) |
| Cu—N₂ | 2.478(1) | 2.094(2) |
| Cu—N₃ | 2.012(1) | 1.990(2) |
| Cu—O₁ | — | 2.359(1) |
| Cu—Cl | 2.2510(4) | 2.2712(5) |
| τ | 0.71^{a} | 0.13^{b} |

^{a}τ₄ calculated according to ref 39.
^{b}τ₅ calculated according to ref 43.

The primary coordination sphere of [CuCl(dpaa^{OMe})]BPh₄ (FIG. 1b) includes axial ligation via the —OMe substituent with a Cu—O1 bond distance of 2.359(1) Å, consistent with a weak interaction. The Cu—N_{pyr} bonds shorten slightly to 1.994(2) Å and 1.990(2) Å while the Cu—N2 contact contracts significantly to 2.094(2) Å upon oxidation (Table 1). This contraction of nearly 0.4 Å is consistent with similar phenomena observed in Cu^{I/II} tripicolylamine systems, and overall, the bond lengths are similar to other reported cupric chloride dipicolylanisidines. The complex has a τ₅=0.13 using the geometry index proposed by Addison and Reedjik for five-coordinate complexes, consistent with a slightly distorted square pyramidal geometry.

CuCl(dpaa^{H}) and CuCl₂(dpaa^{H}) were also crystalized and examined by XRD. CuCl(dpaa^{H}) is a three-coordinate complex with two short Cu—N_{pyr} bonds of 1.965(2) Å and 1.955(2) Å and a Cu—Cl bond of 2.3535(7) A. The N1-Cu—N3 angle of 148° indicates the complex is nearly trigonal planar. In contrast to CuCl(dpaa^{OMe}), the Cu—N2 distance of 2.783(2) Å indicates there is no observable bonding interaction with the aniline nitrogen in the solid state. Attempts to prepare four-coordinate [CuCl(dpaa^{H})⁺ complexes produced five-coordinate bridged species. Mononuclear CuCl₂(dpaa^{H}) has τ₅=0.28 and exhibits a shortened Cu—N2 distance of 2.363(1) Å relative to CuCl(dpaa^{H}). However, this distance is not nearly as short as the Cu—N2 distance observed in [CuCl(dpaa^{OMe})]BPh₄ (2.094(2) Å). The Cu—N2 bond distance is 0.15 Å longer than an analogous cupric dichloride dipicolylanisidine reported in the literature which lacks a para-acyl substituent.

Solution-phase NMR, EPR, and IR Spectroscopies. The solution-phase structures of the Cu^{I} complexes were studied by 600 MHz ¹H-NMR spectroscopy in d₂-DCM. We have previously reported that the CuCl(dpa^{R}) complexes shown in Chart 1b are fluxional in solution and exhibit broadened and deshielded ¹H-NMR spectra relative to the free ligands. Similarly, both CuCl(dpaa^{OMe}) and CuCl(dpaa^{H}) exhibit broadened and deshielded ¹H-NMR spectra, suggesting that these complexes are also fluxional in solution. Additional evidence to this end was obtained by variable temperature NMR (VT-NMR), where the peaks are observed to sharpen upon cooling to 243 K.

X-band EPR spectra collected at 50 K in 1:1 DMF/toluene were examined to investigate the solution-phase structures of the Cu^{II} complexes. [CuCl(dpaa^{OMe})]BPh₄ exhibits an axial spectrum with I=3/2 Cu hyperfine coupling and g-values (g_{⊥}: 2.05, g_{∥}: 2.25) consistent with those previously reported for similar complexes (J. Am. Chem. Soc. 2009, 131, 3230). Superhyperfine coupling from three ¹⁴N I=1 nuclei is also observed along g_{⊥}, indicating a substantial degree of covalence in the ligand field of the Cu^{II} adduct. As might be expected from the solid-state structure, CuCl₂(dpaa^{H}) exhibits a rhombic EPR spectrum.

Solution-phase stretching frequencies of the carbonyl functional groups on the ligands and complexes were collected in DCM and are reported in Table 2. The carbonyl stretching frequencies of dpaa^{OMe} and dpaa^{H} blueshift when Cu^{I} is bound and further blueshift when Cu^{II} is bound, consistent with inductive effects. The two sets of IR data are remarkably similar despite substantial differences in the solid state.

TABLE 2
Carbonyl Stretching Frequencies
of Ligands and Complexes in DCM.

| | ν_{CO} (cm⁻¹) | Δν_{(CO, ligand)} (cm⁻¹) |
|---|---|---|
| dpaa^{H} | 1665 | 0 |
| CuCl(dpaa)^{H} | 1673 | 8 |
| CuCl₂(dpaa)^{H} | 1712 | 47 |
| dpaa^{OMe} | 1670 | 0 |
| CuCl(dpaa^{OMe}) | 1677 | 7 |
| [CuCl(dpaa^{OMe})]BPh₄ | 1713 | 43 |

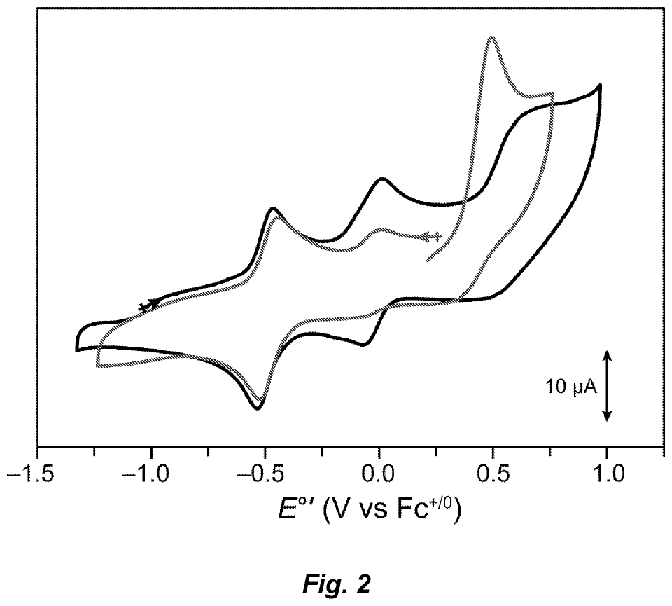

Cyclic Voltammetry (CV). To begin to assess the energetics of CS and CR envisioned to occur photochemically in these complexes (Scheme 1), their electrochemical properties were assessed. CVs of CuCl(dpaa^{OMe}) and [CuCl(dpaa^{OMe})[BPh4] in DMF are shown in FIG. 2. The Cu^{I} complex exhibits three oxidative events. The first of these events occurs at E₁^{∘}′=−0.50 V and is reversible (iₚₐ/iₚₑ≅1 and Eₐ−E_{c}=70 mV, where Eₐ−E_{c}=79 mV for Fc^{+/0}). The second event at E₂^{∘}′=−0.02 V is quasi-reversible (iₚₐ/iₚₑ≅1.5 and Eₐ−E_{c}=95 mV). The third irreversible oxidative event, E₃^{∘}′, occurs at +0.70 V and is assigned to ligand oxidation. For the divalent [CuCl(dpaa^{OMe})][BPh₄], E₂^{∘}′ is significantly diminished and likely becomes increasingly so with slower scan rates in analogy with CuCl(dpa^{OMe}). As was previously observed for CuCl(dpa^{OMe}) and CuCl(dpa^{H}), the first two redox events can be assigned to metal-centered oxidations, both for the Cu^{II/I} couple, but in two different conformations. We have previously ascribed E₁^{∘}′ to arise from the Cu^{II/I} couple in a tetragonal conformation, while E₂^{∘}′ may arise from either a tetrahedral or trigonal conformation. In light of these assignments, the decreased current observed for E₂^{∘}′ in the Cu^{II} complex can be understood by recognizing that in complex with dpaa^{OMe}, the tetragonal conformation is favored for Cu^{II}. The ability of this family of ligands to adopt multiple coordination geometries is critical to the design elements illustrated in Scheme 1 and Chart 1a.

Our control complex, CuCl(dpaa^{H}) exhibits similar electrochemical behavior to CuCl(dpaa^{OMe}) but with an anodic shift in E₁^{∘}′ from −0.50 to −0.36 V (iₚₐ/iₚₑ≅1 and Eₐ−E_{c}=69 mV). Additionally, while E₂^{∘}′ is slightly shifted at −0.05 V, it becomes increasingly irreversible (iₚₐ/iₚₑ≅1.7 and Eₐ−E_{c}=95 mV). E₃^{∘}′ also shifts slightly from 0.70 to 0.63 V. These data suggest that CuCl(dpaa^{H}) has a similar electronic structure to CuCl(dpaa^{OMe}) in solution, making it a good experimental control.

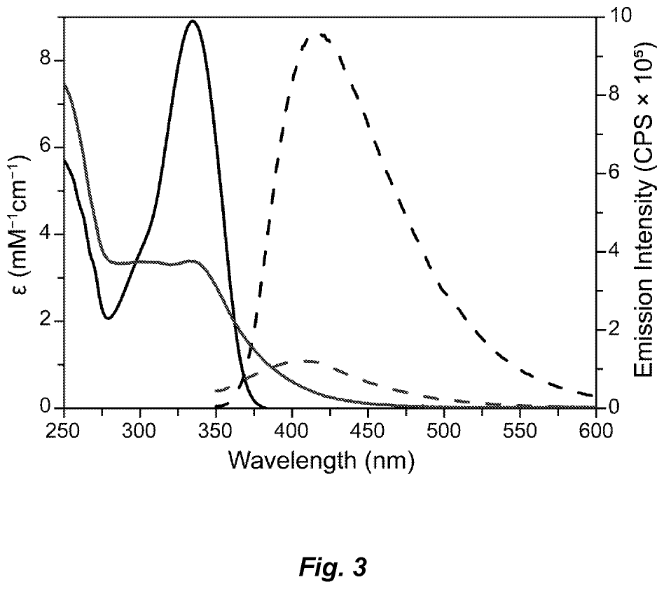

Steady-State Electronic Spectroscopy. The UV-vis absorption and emission spectra of dpaa^{OMe} and CuCl(dpaa^{OMe}) in DCM are shown in FIG. 3. The n_{N}→π* absorbance feature of dpaa^{OMe} occurs at 334 nm, while the same transition occurs at 322 nm for dpaa^{H}. As shown in Table 3, complexation to Cu^{I} does not appreciably shift these features (332 and 321 nm, respectively) but does lower their intensities (E=3390 and 1700 M⁻¹ cm⁻¹, respectively). Both Cu^{I} complexes exhibit unusual peak shapes for these transitions. VT-electronic absorption spectra of CuCl(dpaa^{OMe}) reveal a temperature-dependence of this line shape. We ascribe these observations to the existence of conformational equilibria in our Cu^{I} complexes in solution where the lower-energy state was assigned as having a tetrahedral coordination geometry about Cu^{I}.

TABLE 3
Absorption Maxima and Extinction Coefficients,
Emission Maxima and Quantum Yields.

| | λ_{Abs} (nm) | ε | | |
|---|---|---|---|---|
| | (n_{N}→π*) | (M⁻¹ cm⁻¹) | λ_{Em}^{a} (nm) | Φ_{F}^{a} (%) |
| CuCl(dpaa^{OMe}) | 332 | 3400 | 407 | 0.5 |
| CuCl(dpaa^{H}) | 321 | 1700 | 372 | 1.7 |
| dpaa^{OMe} | 334 | 8900 | 417 | 8.9 |
| dpaa^{H} | 322 | 4300 | 370 | 1.3 |

^{a}λₑₓ is the n_{N}→π* transition for each species.

The emission spectra of both ligands (dpaa^{OMe} and dpaa^{H}) have one prominent feature (FIG. 3). For dpaa^{OMe}, this feature bears a shoulder that redshifts with solvent polarity, consistent with a CT state. In contrast, the emission maximum of dpaa^{H} does not shift with solvent polarity, consistent with a nonpolar excited state. These emissive states have been assigned to overlapping LE and TICT emission bands that are individually resolved in TR-PL experiments (vide infra). The emission maxima for dpaa^{H} and CuCl(dpaa^{H}) are similar at 370 and 372 nm, respectively. In contrast, emission maxima from dpaa^{OMe} and CuCl(dpaa^{OMe}) shift from 417 to 407 nm, respectively. This blueshift suggests destabilization in the presence of the Cu^{I} cation, a result expected if there are significant contributions from a CT excited state. In contrast, the unaffected dpaa^{H} emission maximum is in-line with a nonpolar LE emitting state.

The quantum yields (Φ_{F}, Eq. 1) relative to 9,10-diphenylanthracene for each ligand and complex are presented in Table 3. The Φ_{F} for the free dpaa^{OMe} ligand is 17× higher than Φ_{F} in complex with Cu^{I} (8.9% versus 0.5%, respectively). In contrast, the emission intensity and Φ_{F} of dpaa^{H} increases slightly on complexation to Cu^{I} (from 1.3% to 1.7%, Table 3). As described further in the Discussion section, similar increases have been observed for TICT ligands associated with redox-inert metal cations. Critically for our purposes, the emission quenching observed in CuCl(dpaa^{OMe}) is consistent with ET from a redox-active metal center, as shown in Scheme 1. These findings indicate that the dynamic dpaa^{OMe} ligand is uniquely suited to facilitate Cu^{I}-mediated emission quenching.

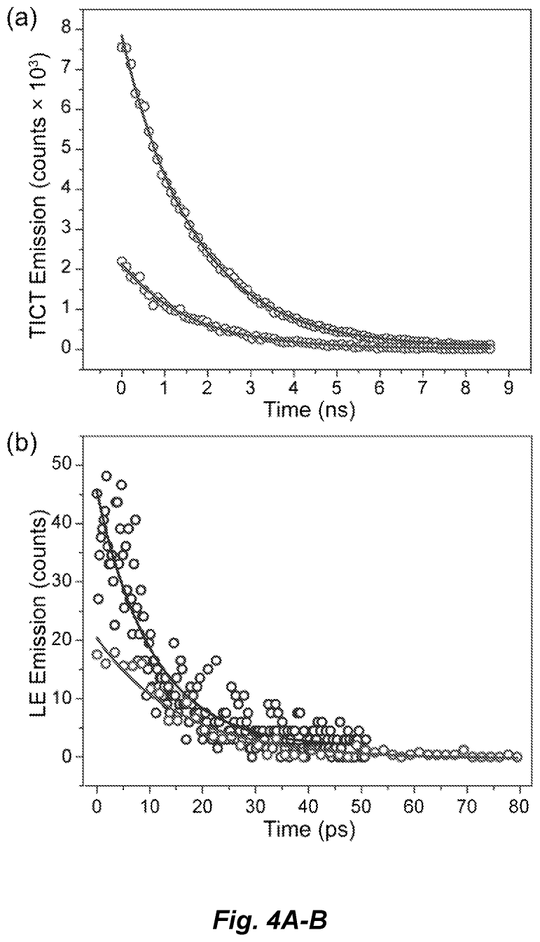

Time-Resolved Photoluminescence (TR-PL) Spectroscopy. The photophysics of ligands and complexes were interrogated by TR-PL. Laser excitation at 325 nm was followed by detection across the full spectral range. The results of these studies are summarized in Table 4, where excited state lifetimes and relative amplitudes are derived from fits to Eq. 2 (FIG. 4), monitoring wavelengths selected to mitigate overlap between emissive states. The complexes and ligands primarily emit from two excited states, LE* and TICT*. A third excited state emits at extremely low intensities and is consistent with ³(π→π*) phosphorescence, as has been seen with other N,N-dialkylaminoacetophenone TICT flurophores (Chem. Phys. Lett. 2009, 481, 78). The higher energy LE* emission band precedes the appearance of the lower energy TICT* feature in all cases. The rates of this LE* to TICT* conversion are in-line with the reported ˜5-10 ps window previously described for this excited state isomerization process (Chem. Phys. Lett. 1987, 137, 408). For both ligands/complexes, the wavelength of LE* emission does not substantially change on complexation of Cu^{I}, but the wavelength of the TICT* emission shifts by 30-40 nm, consistent with previously reported shifts for TICT chromophores ligated to cations at the donor end of the chromophore (Org. Lett. 2001, 3, 1467).

TABLE 4
LE* and TICT* Emission Maxima, Life-
times, and Amplitudes from TR-PL.

| | λ_{LE} | λ_{TICT} | τ_{LE} | τ_{TICT} | % | % |
|---|---|---|---|---|---|---|
| | (nm) | (nm) | (ps)^{a} | (ps)^{a} | A_{LE}^{a} | A_{TICT}^{a} |
| CuCl(dpaa^{OMe}) | 386 | 438 | 34(2) | 1580(40) | 7 | 93 |
| CuCl(dpaa^{H}) | 370 | 440 | 16(1) | 160(20) | 87 | 13 |
| dpaa^{OMe} | 390 | 473 | 23(2) | 1680(20) | 1 | 99 |
| dpaa^{H} | 371 | 474 | 11(1) | 190(20) | 83 | 17 |

^{a}Derived from fits to Eq. 2 with rel. amplitudes corrected for differences in data acquisition times and OD₃₂₅ (λₑₓ = 325 nm).

The emission profiles are ligand-dependent and similar for dpaa^{H}/CuCl(dpaa^{H}) and dpaa^{OMe}/CuCl(dpaa^{OMe}). This similarity can be understood from the fact that the emissive states are principally driven by the ligands themselves—the dpaa^{H} series primarily emits from the LE* state while the dpaa^{OMe} series primarily emits from the TICT* state (see Table 4 for amplitudes). These findings are unsurprising; it is well established that substitution at the ortho-position of TICT fluorophores causes pre-twisting and facilitates access to TICT* excited states. The single-wavelength kinetics of emission decay for each species are displayed in FIG. 4, where the wavelengths monitored were 360 nm (LE*) and 485 nm (TICT*), to minimize overlap from the multiple emissive states and allow us to measure the lifetimes accurately. The excited-state lifetimes determined from these studies are in line with previously reported TICT fluorophores, which generally exhibit LE* lifetimes on the order of ps and TICT* lifetimes on the order of ns. As reflected by the quantum yields provided in Table 3, the relative intensities of the ligands versus the complexes differ for dpaa^{OMe} and are similar for dpaa^{H}. With the quantum yield and excited-state lifetime measurements in hand, we were able to break down the latter into its component radiative and nonradiative contributions. Calculations reveal that the decreased quantum yield of CuCl(dpaa^{OMe}) relative to that of dpaa^{OMe} arises from a decrease in the radiative rate constant for TICT* emission in the presence of the Cu^{I} ion.

Optical Transient Absorption (OTA) Spectroscopy. To investigate the role of the TICT fluorophore in mediating CS, fs OTA experiments were conducted with CuCl(dpa) and CuCl(dpa^{OMe}) control compounds lacking an acyl substituent (Chart 1b). Excitation at 340 nm drives direct MLCT in these complexes and produced excited state absorbance (ESA) features with τ=6.3(7) and 4.3(5) ps according to fits to Eq. 2. In contrast, para-substitution with an acyl group to render the ligands TICT fluorophores leads to ESA features lasting well into the ns regime, several orders of magnitude longer than the controls. These comparatively long-lived states cannot be considered direct MLCT transitions. Instead of exciting an MLCT band, the dpaa^{H} series are excited via n_{N}→π* transitions of the ligand. These ligand-centered excited states subsequently drive ET from Cu^{I} to create the formally MLCT electronic structure described here as the CS state (Scheme 1). Importantly, these complexes are stable to photochemical conditions; samples exhibited deterioration of <2% after exposure to 9×10⁶ laser shots at a power of 400 W as assessed by steady-state UV-vis absorption spectroscopy.

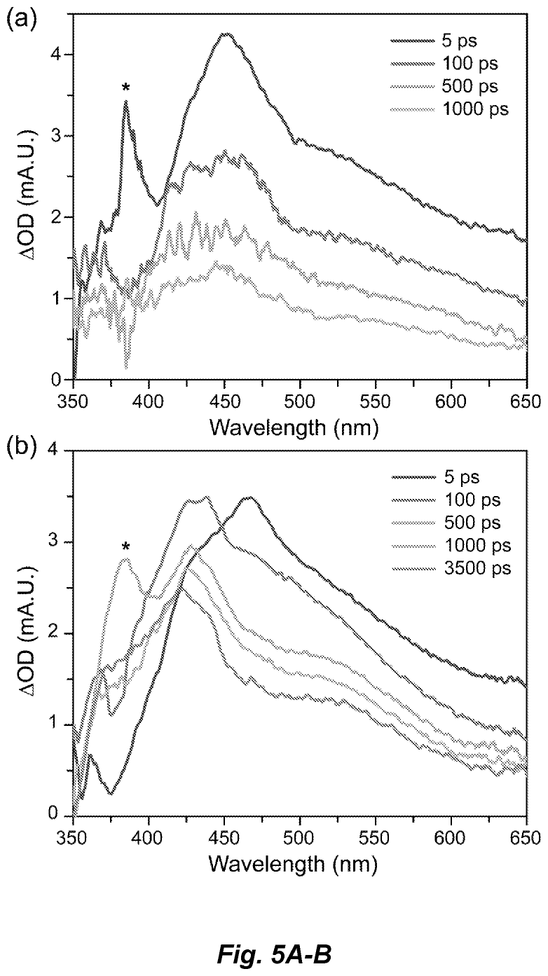

Spectral traces for CuCl(dpaa^{H}) and CuCl(dpaa^{OMe}) from fs OTA experiments are depicted in FIG. 5. CuCl(dpaa^{OMe}) exhibits an ESA feature centered at ˜480 nm that corresponds to the ligand-centered radical anion generated via the TICT* state. Notably, the spectral signature of this ligand radical anion is also the predominant feature of the CS state that may be formed via ET from Cu^{I}(Scheme 1). No discrete ligand-centered radical anion ESA was observed for CuCl(dpaa^{H}), though the existence of a small population may be inferred from the TR-PL data. The OTA spectra of the dpaa^{OMe} ligand alone features an explicit LE* state that precedes the formation of the ligand radical anion and exhibits a lifetime of t=27(1) ps. In contrast for the CuCl(dpaa^{OMe}) complex, the transient associated with the ligand-radical anion is present at the earliest time points measured. The complete decay of this feature in both dpaa^{OMe} and CuCl(dpaa^{OMe}) was not observed within the experimental window (i.e., before 3.5 ns).

For both CuCl(dpaa^{H}) and CuCl(dpaa^{OMe}), the photophysics observed are complicated by additional successor states that arise from the presence of the acyl substituent. In CuCl(dpaa^{OMe}), the ESA at 480 nm converts to another ESA feature centered at 421 nm, that is assigned to a ³(π→π*) state. For CuCl(dpaa^{H}) the ³(π→π*) ESA is centered at 453 nm and lives longer than the 3.5 ns spectral window of the experiment. This state appears to be populated directly from the LE* species rather than via the intermediacy of a TICT* state as in the CuCl(dpaa^{OMe}) system (vide infra). The formation of these ³(π→π*) states occur with rate constants of 1.1×10¹¹ s⁻¹ and 5.0×10¹¹ s⁻¹ for CuCl(dpaa^{OMe}) and CuCl(dpaa^{H}), respectively, and 5.9×10¹⁰ s⁻¹ and 2.5×10¹¹ s⁻¹ for the ligands. Notably, the rate constants associated with the initial rise of the ³(π→π*) excited state in the Cu^{I} complexes are twice those of the unbound ligands. Interestingly, comparing the evolution of the ³(π→π*) ESA for dpaa^{OMe} and CuCl(dpaa^{OMe}) reveals a rise in the former that appears to increase linearly over the fs experiment. In the latter, this rise does not occur.

In order to observe the complete decay of the key ESA features depicted in FIG. 5, ns OTA experiments were undertaken. The lifetimes obtained from these studies. Unfortunately, the decay of ligand radical anion states for either dpaa^{OMe} or CuCl(dpaa^{OMe}) was complete by the beginning of our experimentally observable time window (which starts at 10 ns), indicating that the decay of this feature is complete between 3.5 and 10 ns. The ns OTA spectra do reveal the existence of a third excited state for both complexes that presumably arises from the ³(π→π*) state. These ESA features are centered at 407 and 416 nm for CuCl(dpaa^{OMe}) and CuCl(dpaa^{OMe}) for this new state, respectively. We assign these states to n_{O}→π* excited state transitions, in accordance with literature precedent for acyl-substituted TICT fluorophores. Interestingly, the lifetimes of these highest energy ESA features appear to be more dependent on the presence of Cu than on the ligand itself. The ligands dpaa^{H} and dpaa^{OMe} have similar lifetimes (2700±320 and 2600±150 ns, respectively) while CuCl(dpaa^{H}) and CuCl(dpaa^{OMe}) also exhibit similarities (420±20 and 460±20 ns, respectively). This excited state reportedly decays via internal conversion and lasts longer than weak phosphorescence observed by TR-PL.

### Discussion

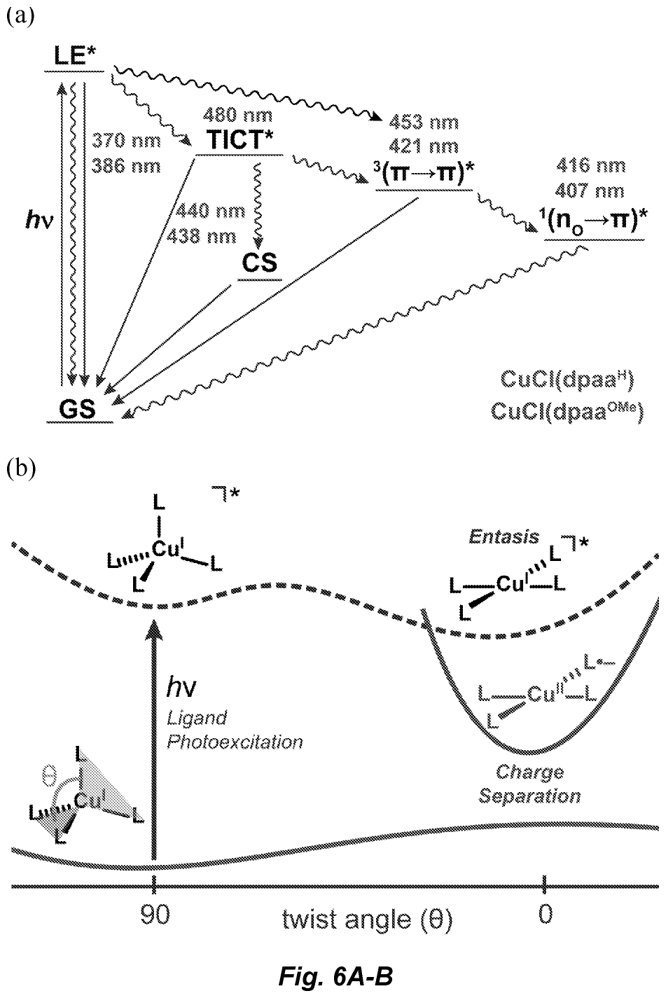

A model for the photophysical dynamics of CuCl(dpaa^{OMe}) and CuCl(dpaa^{H}) is presented in FIG. 6. Excitation of the n_{N}→π* transition of the aniline ligand results in the prompt formation of LE* excited states for both CuCl(dpaa^{H}) complexes. In CuCl(dpaa^{OMe}), LE* rapidly converts to the desired TICT* state while for CuCl(dpaa^{H}), no TICT* state is observed via fs OTA spectroscopy. However, a low intensity emissive state observed by TR-PL is consistent with a small population of the TICT* excited state for CuCl(dpaa^{H}). Specifically, the energy of this feature is consistent with that observed in CuCl(dpaa^{OMe}) (440 versus 438 nm), it blueshifts with metal binding (from 474 to 440 nm), and its' lifetime is longer than the observed LE* state (160 versus 16 ps, Table 4).

The diminished Φ_{F} of CuCl(dpaa^{OMe}) relative to dpaa^{OMe} results from a decrease in the rate of radiative deactivation for this complex relative to its free ligand. With kᵣ proportional to the square of the transition dipole moment (μ) and the cube of the energy gap between radiatively coupled states (ΔE), several explanations for this observation are possible. The energy gap could be implicated, as a decrease in kᵣ could result from a decreasing ΔE. However, blue-shifted emission from the complex relative to the ligand suggests that changes in the energy gap cannot account for the observed differences in kᵣ. This leaves μ, where a decrease in the transition dipole moment consequently decreases the observed kᵣ. Factors affecting p could include structural reorganization, solvent relaxation, and modulation of the excited state charge distribution, all of which are known to be at play in TICT fluorophores. These findings suggest that ET from Cu^{I} does not compete with radiative deactivation of the excited fluorophore. This surprising result led us to perform an estimate for the driving force for CS from the TICT* excited state. The details rely on the established theoretical framework of TICT fluorophore energetics (Pure Appl. Chem. 1983, 55, 245). Our conclusion from these investigations is that we likely have a very low driving force for CS from the TICT* state. With a system designed to lower the reorganization energy of CS and raise it for CR, the precise balance of driving force and reorganization energy in these steps will exert a great impact on the ET rate constants observed. Modified systems in which these energetics are tipped in favor of formation of discrete and observable long-lived CS states are being prepared.

A consequence of employing ligand-centered excited states as the direct precursor to our Cu^{II}-L^{•-} CS state is that the photophysics of the ligand centered states will impact the quantum yield of CS. Here our design is inhibited by the photophysics of aromatic carbonyls, where the carbonyl enables intersystem crossing into unproductive ³(π→π*) and successor excited states incapable of further participation in ET with Cu^{I}. This off-pathway reactivity limits the maximum possible quantum yield for CS in these systems and must be eliminated to improve efficiency. Both the ³(π→π*) and n_{O}→π* states are observed by OTA spectroscopy and the latter represents the longest living transient observed. Subsequent designs will employ electron-withdrawing moieties capable of increasing the quantum yield of the desired ligand-centered TICT* excited state.

We do not observe evidence for a CS state with CuCl(dpaa^{H}) by OTA spectroscopy. Instead, our analysis supports conversion of the strongly emitting LE* state directly to the weakly emitting ³(π→π*) state as shown in FIG. 6. This precursor-successor relationship has been previously observed in other acetophenone derivatives, where both TICT* and LE* were shown to populate the analogous ³(π→π*) state. While similar phenomena are observed for CuCl(dpaa^{OMe}) and dpaa^{OMe}, the emission spectra of these species is dominated by the TICT* excited state and quenching via ET to form the CS state in FIG. 6 is thought to be the primary Cu^{I}-mediated quenching mechanism in this case. Notably, in the absence of Cu, the dpaa^{OMe} ligand gives rise to the ³(π→π*) with an initial burst, followed by a slow linear phase. We ascribe the burst to formation via the LE* state, and the linear phase to formation via the TICT* state. In the presence of Cu^{I}, no linear phase is observed, presumably due to the conversion to a new dark state, namely the CS state, which forms preferentially over ³(π→π*) and is primarily responsible for the observed fluorescence quenching of CuCl(dpaa^{OMe}) relative to dpaa^{OMe} The rapid decay of CuCl(dpa^{H}) and CuCl(dpa^{OMe}) in the fs regime reveals the necessity of the electron-withdrawing acyl group, and in turn, the presence of the strategically placed TICT axis, in the generation of transient CS states exceeding ˜6 ps. The twisting of this TICT* state is intended to destabilize Cu^{I} and favor the formation of Cu^{II} by forcing the bound metal ion into a tetragonal geometry (representing a transient entatic state). These ligand-induced conformational dynamics promote reduction of the aniline radical cation of the TICT* state with the formation of square pyramidal Cu^{II}. The time-resolved spectroscopic data presented above support this mechanism for CuCl(dpaa^{OMe}) and suggest that the design principles outlined in Chart 1a provide a novel mechanism for the generation of extended CS lifetimes in a first-row transition metal complex that is stable under photochemical conditions. Additionally, these findings demonstrate that the CS states reported here are not photoinduced MLCTs from Cu^{I} to diimine ligands, as have been widely studied by others (J. Am. Chem. Soc. 2003, 125, 7022). While Cu^{I}-pyridine adducts have MLCTs within this region of the electromagnetic spectrum, the resultant MLCT excited states should not have any dependence on the acetophenone moiety present in our system.

### Conclusions

The key findings from these studies were the following: a) Incorporation of a TICT (twisted intramolecular charge transfer) fluorophore increases excited state lifetimes by >10³. b) The presence of the ortho-OMe group results in the formation of a ligand-centered radical anion that lives 7-12 ns while without it, the same state vanishes by 1 ns. c) Our Cu(I) complexes are extremely robust to photophysical conditions, decaying only ˜3% after exposure to 107 laser shots. Compared with [Cu(phen)₂]⁺ complexes which decay rapidly under photophysical conditions. We hypothesize that the conformational dynamicity of our complexes, and the resultant ability to accommodate both the Cu(I) state and the transiently formed Cu(II) state, makes our systems extremely robust to photophysical conditions.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Materials and Methods

General Considerations. Unless otherwise noted, all manipulations were carried out under anaerobic conditions using standard Schlenk line and glovebox techniques. A glovebox in which oxygen is excluded but water is present was employed for all manipulations except electrochemical experiments, which were performed in a dry (water-free) glovebox.

Materials. All chemicals were used as received from the following suppliers and used without further purification unless otherwise specified: toluene, hexanes, pentane, dimethylformamide (DMF), ethyl acetate (EtOAc), dichloromethane (DCM), and Fe metal powder were obtained from Fisher Scientific; 2-pyridinecarboxaldehyde and 4-aminoacetophenone were obtained from TCI; sodium trisacetoxyborate was obtained from Oakwood Chemical; 4-amino-3-methoxyacetophenone was obtained from Ambeed; NH₄Cl and Na₂CO₃ were obtained from VWR; NaHCO₃ was obtained from Merck; Na₂SO₄ was obtained from Alfa Aesar; and tosylic acid and basic alumina were obtained from Sigma. All deuterated solvents were from Cam-bridge Isotope Laboratory. Dry solvents were obtained from a Jorg C.

### Meyer Solvent Purification System.

Acetonitrile (MeCN), methanol (MeOH), pentane, dimethylformamide (DMF), dichloromethane (DCM), obtained from Fisher Scientific were purified further using a Jorg C. Meyer Solvent Purification System in which hydrocarbon and ethereal solvents were sparged with nitrogen before being deoxygenated and dried by passage through Q5 and activated alumina columns, respectively. Halogenated solvents were sparged with nitrogen and passed through two activated alumina columns. CuCl₂(H₂O)₂ was obtained from Fisher Scientific, NaBPh₄ from Strem, and all deuterated solvents were from Cambridge Isotope Laboratory. CuCl was obtained from Mallinckrodt, was stirred in acetic acid and subsequently washed with ethanol and diethyl ether before use. Ferrocene (Fc⁰) was purified by recrystallization and [Bu₄N][PF₆](Oakwood Chemicals) was recrystallized thrice from ethanol and dried under vacuum.

Physical Methods and Instrumentation. ¹H- and ¹³C-NMR data were collected on a Bruker spectrometer operating at 600 MHz at ambient temperatures or a Carver B500 spectrometer operating at 500 MHz for variable-temperature experiments. Chemical shifts are referenced to residual solvent peaks: CDCl₃ (δ 7.26 ppm for ¹H and δ 77.16 for ¹³C), CD₂Cl₂ (δ 5.32 ppm for ¹H and δ 53.84 for ¹³C), (CD₃)₂SO (δ 2.50 ppm for ¹H and δ 39.52 for ¹³C). Steady-state UV-visible absorption spectra were obtained on an Agilent Technologies 8454 spectrophotometer at ambient temperature unless otherwise specified. Perpendicular-mode X-band EPR spectra were collected using a Bruker EMX spectrometer equipped with a ER041XG microwave bridge using the following spectrometer settings: attenuation=30 dB, microwave power=0.2 mW, frequency=9.35 GHz, modulation amplitude=1.0 G, gain=30 dB, conversion time=11.25 ms, time constant=10.24 ms, sweep width=2000 G and resolution=4000 points. EPR spectra were modelled using EasySpin/Matlab. Fourier transform-infrared (FT-IR) spectra were recorded on a PerkinElmer Spectrum 100 spectrometer using a CaF₂ cell. High-resolution mass spectra (HR-MS) were obtained on a Waters Synapt G2-Si ESI instrument at the UIUC School of Chemical Sciences Mass Spectrometry Laboratory. CHN elemental analyses were collected on an Exeter Analytical CE 440 instrument in the UIUC School of Chemical Sciences Microanalysis Laboratory. Steady-state emission spectra were obtained on an ISS Chronos FD Fluorescence Lifetime Spectrophotometer with 1 mm slits at ambient temperature. Fluorescence quantum yields relative to 9,10-diphenylanthracene as reference (Φ_{F}=0.90 when excited between 275-405 nm and collected between 380-550 nm in cyclohexane) were calculated according to Eq. 1, where subscripts S and R indicate sample or reference, I the area under the curve, f the optical density at λₑₓ, and n the refractive index.

$${Φ}_{F} = {Φ}_{R} \{ \frac{{I}_{S} {f}_{R} {n}_{S}^{2}}{{I}_{R} {f}_{S} {n}_{R}^{2}} \} \qquad ( 1 )$$

Electrochemistry Measurements. Electrochemical experiments were performed on a Pine Wavedriver 10 potentiostat using a 3.0 mm glassy carbon working electrode, a Pt wire auxiliary electrode, and a Ag wire pseudo-reference electrode. Data acquisition was carried out at ambient temperature (20-24° C.) in a nitrogen-filled glovebox for solution samples containing 1.0 mM of analyte and 100 mM of [Bu₄N][PF₆] supporting electrolyte dissolved in dry, degassed DMF. All potentials were referenced to Fc^{+/0} by adding Fc⁰ as an internal standard at the end of each experimental run, and all scans were initiated from open-circuit potential (OCP).

Single Crystal X-ray Diffraction (XRD). XRD data were collected on a Bruker D8 Venture kappa diffractometer equipped with a Photon II CPAD detector. An I μs microfocus source provided the Mo Kα radiation (λ=0.71073 Å) that was monochromated with multilayer mirrors. The collection, cell refinement, and integration of intensity data were carried out with the APEX3 software. Multi-scan absorption corrections were performed numerically with SADABS. The initial structure solution was solved with either intrinsic phasing methods SHELXT or direct methods and refined with the full-matrix least-squares SHELXL program within the OLEX2 GUI.

Time-Resolved Spectroscopy. Optical Transient Absorption (OTA) spectra and kinetics measurements were carried out at the Center for Nanoscale Materials (CNM) at Argonne National Laboratory using an amplified Ti:sapphire laser system (Spectra Physics, Solstice Ace) and an automated data acquisition system (Ultrafast Systems, HELIOS). The amplifier produced 800 nm, 100 fs pulses at 5 kHz. Ninety percent of the output from the amplifier was split and used to pump an optical parametric amplifier (TOPAS) which provided the excitation pulses. The TA system enables three-dimensional data collection (spectra/time/AOD).

Continuum probe pulses were generated by sending the remaining 10% output from the laser amplifier down a computer controlled optical delay line then focusing it into a 3 mm thick piece of sapphire crystal. The residual 800 nm light was removed from the probe beam with an interference notch filter leaving a 440-750 nm white-light continuum. The probe beam was then focused into a stirred 1 mm quartz cuvette containing the solution to be measured. The transmitted probe beam was detected using a fiber optically coupled spectrograph with a 1D, 2048-pixel CCD array detector.

The excitation beam (0.16-0.32 J/pulse) was depolarized then overlapped on the probe beam spot on the sample at an incident angle of 5° after being optically chopped at 2.5 Hz using a synchronous chopper so that the spectrograph measured the transmitted probe beam alternatively as T_{ON} and T_{OFF}. The differential extinction ΔA=−log₁₀(T_{ON}/T_{OFF}) was calculated for each pair of pulses and was typically averaged over a two second interval for each delay time. Temporal chirp in the probe pulse was measured and corrected for by making a measurement on neat solvent; the resulting signal was then fitted for each probe wavelength to determine the zero-delay position between pump and probe. For longer time scale processes, the probe light comes from a continuum light source (Ultrafast Systems, EOS). In this case, the system operates at 1 kHz and has a time resolution of 200 ps/point.

TR-PL was performed using a 35 fs, 2 kHz amplified Ti:sapphire laser (Spectra Physics) that pumped an optical parametric amplifier to produce 325 nm pump pulses. PL was collected with a quartz lens, passed through a long pass filter, dispersed in a 0.15 m spectrograph and detected with a single-photon sensitive streak camera detector (Hamatsu).

Samples for both OTA and TR-PL were prepared in DCM in an inert atmosphere glovebox. Bulk solutions were prepared to have equivalent optical densities of 0.5 at λ=340 nm. Experiments were paused after every 9×10⁶ laser shots at 400 μW and assessed for decomposition via steady-state UV-vis absorption spectroscopy. We observed <2% sample decomposition for both Cu^{I} complexes over this time, while ligands decompose by about 14% under the same conditions. fs OTA spectra were corrected for group velocity dispersion (GVD) using Surface XPlorer software and TR-PL measurements corrected for shifting t=0 in picosecond measurements. All kinetics were fit to Eq. 2 for a monoexponential decay/rise out to five lifetimes using OriginLab 2020. The variables A, and y₀ are constants, y corresponds to AOD or emission counts, and τ the excited state lifetime.

$$y = {y}_{0} + A {e}^{- t / τ} \qquad ( 2 )$$

### Example 2. Synthesis of Coordination Complex

CuCl(dpaa^{OMe}). In a Schlenk flask under N₂ dpaa^{OMe} (112 mg, 0.32 mmol) was dissolved in MeCN (˜3 mL) and was added to a stirring suspension of CuCl (35 mg, 0.35 mmol) in MeCN (˜3 mL). The dark yellow solution was heated to reflux for 15 min, cooled to room temperature (rt), and allowed to stir overnight. The solution was filtered through celite using a medium porous fritted funnel and washed with MeCN (15 mL). The solvent was evaporated under reduced pressure and the remaining residue was dissolved in DCM (˜2 mL). Pentane was added to induce precipitation of a light-yellow solid, which was washed with pentane and dried under reduced pressure to afford CuCl(dpaa^{OMe}) (93 mg, 65%). Yellow crystals that were suitable for single crystal XRD were obtained by layer diffusion of pentane into DCM at −36° C. ¹H-NMR (d₆-DMSO, 600 MHz): 8.57 (s, 2H), 7.91 (t, J=7.26 Hz, 2H), 7.58 (d, J=7.36 Hz, 2H), 7.46-7.35 (m, 4H), 7.21 (d, J=7.79 Hz, 1H), 4.54 (s, 4H), 3.70 (s, 3H), 2.47 (s, 3H). IR (solution in DCM): 3067, 3044, 2991, 2967, 1677, 1597, 1571, 1507, 1480, 1460, 1444, 1416, 1386, 1358 cm⁻¹. HRMS: ESI Positive ion mode m/z [M⁺] calcd. for: 445.0600, found: 445.0625. Anal. calcd. for: C₄₂H₄₂Cl₃Cu₃N₆O₄: C, 50.86; H, 4.27; N, 8.47, found: C, 50.98; H, 4.66; N, 8.09. [CuCl(dpaa^{OMe})]BPh₄. dpaa^{OMe} (50 mg, 0.14 mmol) was dissolved in a minimal amount of MeOH (˜1 mL) in a vial equipped with stirbar and condenser. CuCl₂(H₂O)₂ (27 mg, 0.16 mmol) was dissolved in water (1 mL) and added dropwise to the stirred solution of ligand. This solution was allowed to stir for 3 hours at which point NaBPh₄ (192 mg, 0.56 mmol) was added to immediately precipitate the product as a pale green solid (102 mg, 83%), which was isolated via vacuum filtration. Vapor diffusion with DCM and pentane antisolvent at −36° C. afforded turquoise crystals that were suitable for XRD. EPR (DMF/toluene 1:1) g_{⊥}: 2.05, g_{∥}: 2.25, A_{∥}168×10⁻⁴ cm⁻¹. IR (solution in DCM): 3076, 3061, 3047, 3037, 3002, 2986, 1751, 1713, 1612, 1582, 1503, 1479, 1460, 1444, 1414, 1364 cm⁻¹. HRMS: ESI Positive ion mode m/z [M⁺] calcd. for: 445.0618, found: 445.0618. Anal. calcd. for C₄₅H₄₁BClCuN₃O₂·0.75CH₂Cl₂: C, 66.26; H, 5.17; N, 5.07, found: C, 66.33; H, 5.11; N, 5.19.

CuCl(dpaa^{H}). To a vial equipped with a stirbar under N₂, dpaa^{H} (192 mg, 0.61 mmol) dissolved in dry MeCN (1 mL, 0.16 M), and then CuCl (60 mg, 0.61 mmol) was added. This mixture was heated to reflux and subsequently allowed to cool overnight. The solid thus formed was filtered to afford CuCl(dpaa^{H}) as a yellow crystalline solid (139 mg, 55%). These crystals were suitable for XRD. ¹H-NMR (d₆-DMSO, 600 MHz): 8.60 (s, br, 2H), 7.93 (s, br, 2H), 7.70 (s, br, 2H), 7.55 (s, br, 2H), 7.43 (s, br, 2H), 6.72 (s, br, 2H), 4.91 (s, 4H), 2.38 (s, 3H). IR: 3076, 3068, 3048, 2975, 1673, 1601, 1572, 1519, 1483, 1440, 1393, 1360 cm⁻¹. HRMS: ESI Positive ion mode m/z [M⁺] calcd. for: 415.0513, found: 415.0518. Anal. calcd. for: C₂₀H₁₉ClCuN₃O·26H₂O·0.12MeCN: C, 57.07; H, 4.70; N, 10.26, found: C, 56.94; H, 4.55; N, 10.26.

CuCl₂(dpaa^{H}). The supernatant from the CuCl(dpaa^{H}) synthesis in MeCN was allowed to sit for 4 days, at which point isolable green crystals suitable for X-ray structural analysis were obtained. Alternatively, dpaa^{H} (100 mg, 0.32 mmol) could be dissolved in minimal MeOH under N₂, and CuCl₂(H₂O)₂ (59 mg, 0.35 mmol) dissolved in minimal water (2 mL) added dropwise to this dpaa^{H} solution. The solution was placed in the freezer overnight and afforded turquoise crystals after two days (47 mg, 33%). EPR (DMF/toluene 1:1) g₁: 2.01, g₂: 2.12, g₃: 2.24, A₁: 33×10⁻⁴ cm⁻¹, A₂: 50×10⁻⁴ cm⁻¹, A₃: 122×10⁻⁴ cm⁻¹. IR (solution in DCM): 3066, 3054, 1752, 1712, 1675, 1610, 1578, 1428, 1362 cm⁻¹. HRMS: ESI Positive ion mode m/z [M−Cl⁻]calcd. for: 416.3880, found: 416.0514. Anal. calcd. for C₂₀H₁₉Cl₂CuN₃O·0.14H₂O·0.24MeOH: C, 52.61; H, 4.42; N, 9.09, found: C, 52.61; H, 4.43; N, 9.09.

### Example 3. Synthesis of Dpaa^{H} Ligands

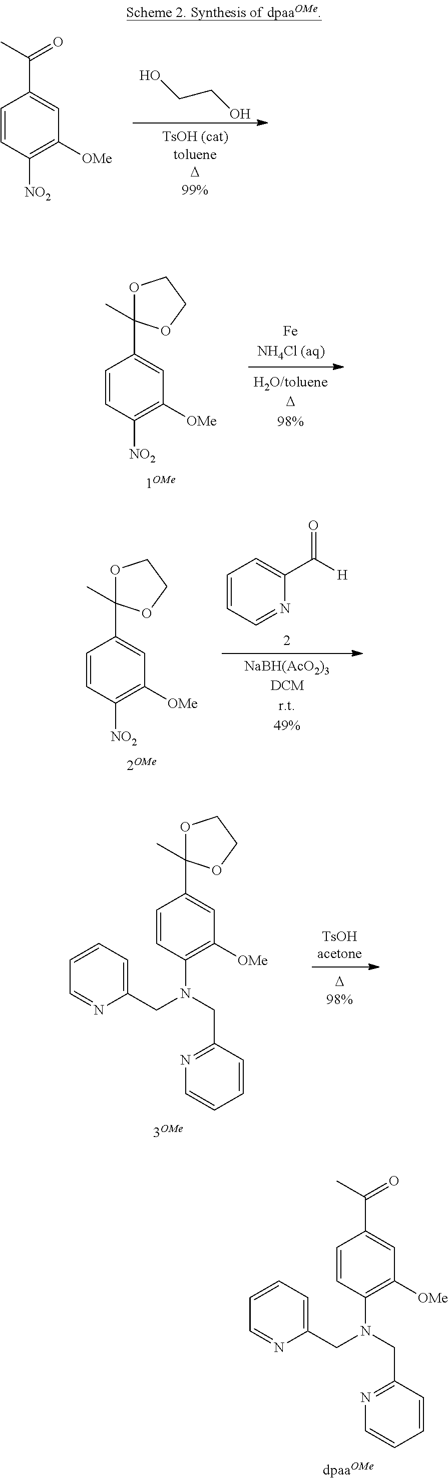

I^{OMe}. 3-methoxy-4-nitroacetophenone (449 mg, 2.30 mmol) was added to a round bottom flask equipped with stirbar, condenser, and Dean-Stark trap under N₂. This was dissolved in toluene (11 mL, 0.20 M) and ethylene glycol (0.390 mL, 6.90 mmol), then tosylic acid (44 mg, 0.23 mmol) was added and the reaction heated to reflux and allowed to proceed overnight. The reaction was extracted with saturated NaHCO₃ and the aqueous layer rinsed with DCM. The organic layers were combined, dried over Na₂SO₄, and concentrated under reduced pressure to afford 1^{OMe} as a white solid (548 mg, 99%). ¹H NMR (CDCl₃, 600 MHz): 7.82 (d, J=8.21, 1H), 7.22 (d, J=1.55 Hz, 1H), 7.14 (dd, J₁=8.36 Hz, J₂=1.57 Hz), 4.09-4.05 (m, 2H), 3.98 (s, 3H), 3.79-3.76 (m, 2H), 1.64 (s, 1H). ¹³C NMR (CDCl₃, 151 MHz): 153.1, 150.7, 139.2, 126.0, 117.4, 110.6, 108.3, 64.8, 56.7, 27.6. HRMS: ESI Positive ion mode m/z [M+1] Calculated. 240.0827 Observed. 240.0870.

2^{OMe}. To a round bottom flask equipped with stirbar and condenser under N₂ was added 1^{OMe} (548 mg, 2.29 mmol), which was subsequently dissolved in toluene (29 mL, 0.08 M). Fe powder (640 mg, 11.45 mmol) and then aqueous NH₄Cl (26 mg, 0.5 mmol in 0.5 mL H₂O) were added and the reaction was allowed to reflux for 3 hours. The reaction mixture was then filtered over celite, rinsed with toluene, dried over Na₂SO₄, and concentrated under reduced pressure to afford 2^{OMe} as yellow solid (470 mg, 98%). ¹H NMR (CDCl₃, 600 MHz): 6.92-6.88 (m, 2H), 6.67 (d, J=8.51 Hz, 1H), 4.05-3.98 (m, 2H), 3.86 (s, 3H), 1.65 (s, 3H). ¹³C NMR (CDCl₃, 151 MHz): 147.1, 135.8, 133.7, 118.0, 114.5, 109.1, 107.8, 64.5, 55.7, 27.9. HRMS: ESI Positive ion mode m/z [M+1] Calculated. 210.1085 Observed. 210.1136.

3^{OMe}. 2^{OMe}(470 mg, 2.25 mmol) was added to a vial under N₂ and dissolved in DCM (2.50 mL, 0.9 M). 2-pyridinecarboxaldehyde (0.855 mL, 8.98 mmol) was added and the reaction allowed to stir for 30 minutes, at which point STAB (1.904 g, 8.98 mmol) was added and the reaction allowed to stir overnight. The organic layer was then extracted with Na₂CO₃ and the aqueous layer rinsed with DCM. These organic extracts were combined, dried over Na₂SO₄, and concentrated under reduced pressure to afford a golden-brown oil, which was subjected to flash chromatography (7:3 hexanes/EtOAc on basic alumina, R_{f}=0.06) to afford 3^{OMe} as an off-white solid (433 mg, 49%). ¹H NMR (CDCl3, 600 MHz): 8.49 (d, J=4.87 Hz, 2H), 7.58 (td, J₁=7.73 Hz, J₂=1.60 Hz, 2H), 7.50 (d, J=7.73 Hz), 7.09 (td, J₁=6.15 Hz, J₂=1.61 Hz, 2H), 6.98 (s, 1H), 6.85 (s, 2H), 4.51 (s, 4H), 4.02-3.98 (m, 2H), 3.84 (s, 3H), 3.81-3.77 (m, 2H), 1.62 (s, 3H). ¹³C NMR (CDCl₃, 151 MHz): 159.9, 152.4, 149.0, 139.1, 137.2, 136.5, 122.3, 121.8, 118.8, 117.6, 108.9, 64.6, 58.7, 55.6, 27.6. HRMS: ESI Positive ion mode m/z [M+1] Calculated. 392.1929 Observed. 392.1973.

dpaa^{OMe}. In a round bottom flask equipped with stirbar and condenser 3^{OMe} (433 mg, 1.11 mmol) was dissolved in acetone (10 mL, 0.1 M). Tosylic acid (419 mg, 2.43 mmol) was added and the reaction heated to reflux for 30 minutes. The resulting solution was cooled to room temperature and extracted with DCM and saturated Na₂CO₃. The organics were dried over Na₂SO₄ and concentrated under reduced pressure to afford dpaa^{OMe} as a white solid (378 mg, 98%). Crystals suitable for XRD can be grown via vapor diffusion of pentane into a solution of DCM at −20° C. ¹H NMR (d-DMSO, 600 MHz): 8.48 (dq, J₁=4.78 Hz, J₂=0.82 Hz, 2H), 7.72 (td, J₁=7.75 Hz, J₂=1.83 Hz, 2H), 7.42-7.39 (m, 2H), 7.37 (d, J=7.86 Hz, 2H), 7.23 (ddd, J₁=7.62 Hz, J₂=4.92 Hz, J₃=0.85 Hz, 2H), 6.82 (d, J=8.98, 1H), 4.64 (s, 4H), 3.79 (s, 3H), 2.45 (s, 3H). ¹³C NMR (d-DMSO, 151 MHz): 196.5, 159.1, 151.1, 149.4, 144.2, 137.1, 130.0, 123.2, 122.6, 122.2, 118.2, 111.3, 58.1, 56.1, 26.7. IR (solution in DCM): 3081, 3064, 3049, 3038, 3013, 2989, 2968, 2940, 1670, 1592, 1570, 1510, 1474, 1465, 1435, 1417, 1391, 1356 cm⁻¹. HRMS: ESI Positive ion mode m/z [M+1] Calculated. 348.1667 Observed. 348.1708.

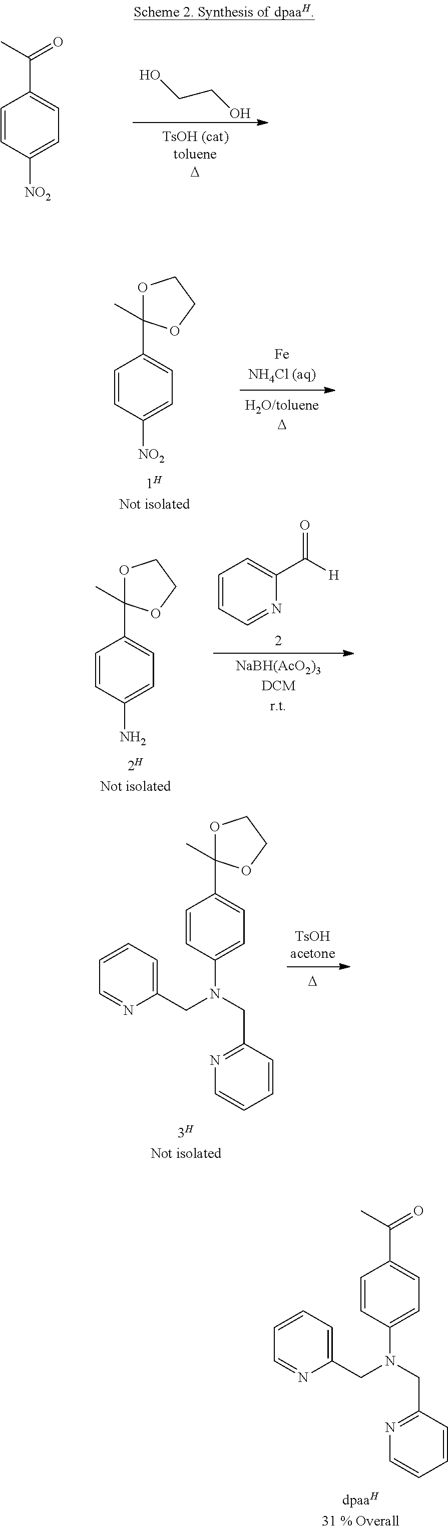

dpaa^{H}. The synthesis of dpaa^{H} is procedurally identical to that of dpaa^{OMe}, except that 4-nitroacetophenone is used in place of 3-methoxy-4-nitroacetophenone in the first step. Discrete intermediates were challenging to isolate at scale due to the acetal's lability. Telescoping from 1^{H} to dpaa^{H} affords the ligand as a white powder, after chromatography (7:3 hexanes/EtOAc on basic alumna, R_{f}=0.03), in 31% overall yield over 4 steps. ¹H NMR (d-DMSO, 600 MHz): 8.57 (dq, J₁=4.73 Hz, J₂=0.77 Hz, 2H), 7.75 (td, J₁=7.68 Hz, J₂=1.80 Hz, 2H), 7.70 (d, J=9.07, 2H), 7.31-7.26 (m, 4H), 6.71 (d, J=9.19 Hz, 2H), 4.94 (s, 4H), 2.38 (s, 3H). ¹³C NMR (d-DMSO, 151 MHz): 195.7, 158.5, 152.1, 149.9, 137.3, 130.6, 125.9, 122.8, 121.6, 111.8, 57.1, 26.5. IR (solution in DCM): 3066, 3048, 3015, 2992, 2981, 2926, 1665, 1600, 1592, 1573, 1556, 1523, 1474, 1432, 1396, 1359 cm⁻¹. HRMS: ESI Positive ion mode m/z [M+1] Calculated. 318.1562 Observed. 318.1605.

### Example 4. Modifications and Applications

The disparate time scales of photodriven charge separation (˜fs) and steps in chemical reactions (˜μs) represent an inherent bottleneck in solar-to-fuels technology. To solve this problem, we have created molecular rectifiers that undergo light-induced conformational changes that speed up charge separation, and slow down the reverse process, charge recombination. These dynamics create a charge separated state that lives long enough to facilitate chemical catalysis. Inspired by nature, these principles mimic a mechanism that is common in nature but rarely applied in synthetic and artificial systems.

Applications include the efficient conversion of solar energy to electrical, chemical, and chemical potential energies. The optoelectronic molecular rectifiers we have created can be applied in porous materials, embedded into membranes, coated onto surfaces, and used as homogeneous photosynthetic catalysts.

For example, applying molecular rectifiers in a surface array can enable highly efficient photocurrent generation that could find application in high performance instrumentation operating under low light conditions where every photon counts. The disclosed molecular rectifiers also represent a new paradigm in solar technology and solar-to-electrical conversion will allow us to compare with commercial systems currently available. Modification of the acceptor sides of our molecular rectifiers with CoCl(diglyoxime) will create pyridyl TICT ligands for a hydrogen evolution reaction (HER) catalyst, as described in Scheme 3.

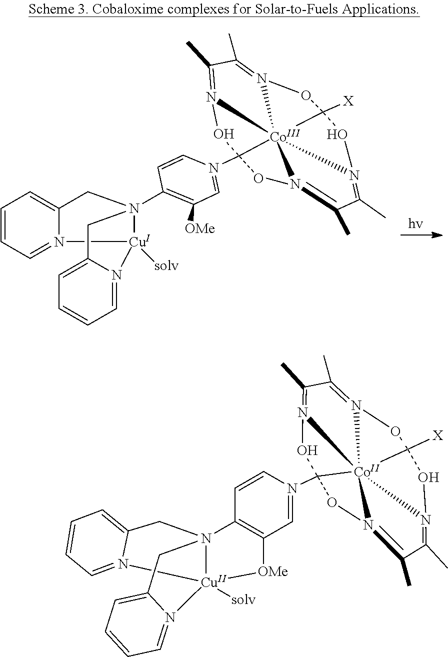

Another way to extend the applicability of the designs presented here is through incorporation of proton-transfer elements, such as incorporating a secondary metal site that undergoes proton-coupled oxidation via an axial ligand capable of facilitating intervalence charge transfer (IVCT). Such a system would use light to drive the formation of a proton concentration gradient, mimicking the mechanism by which natural photosynthetic systems operate (Scheme 4).

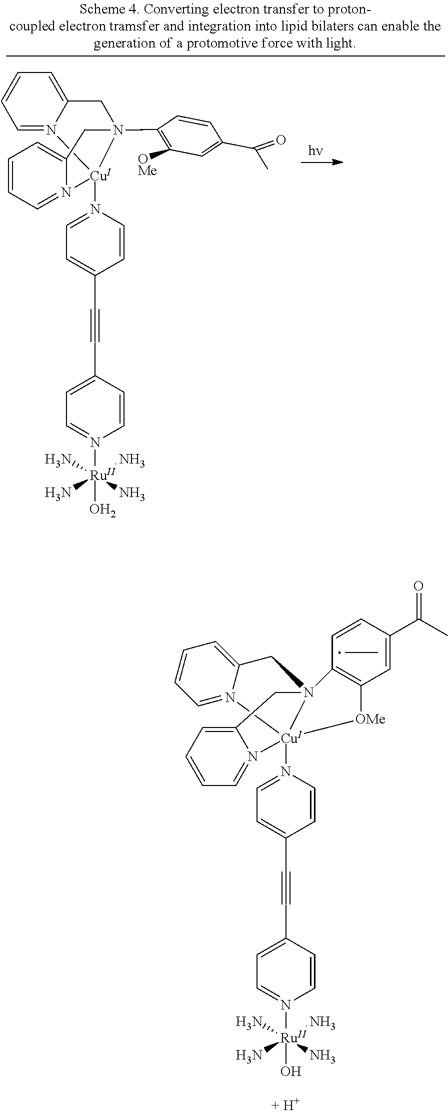

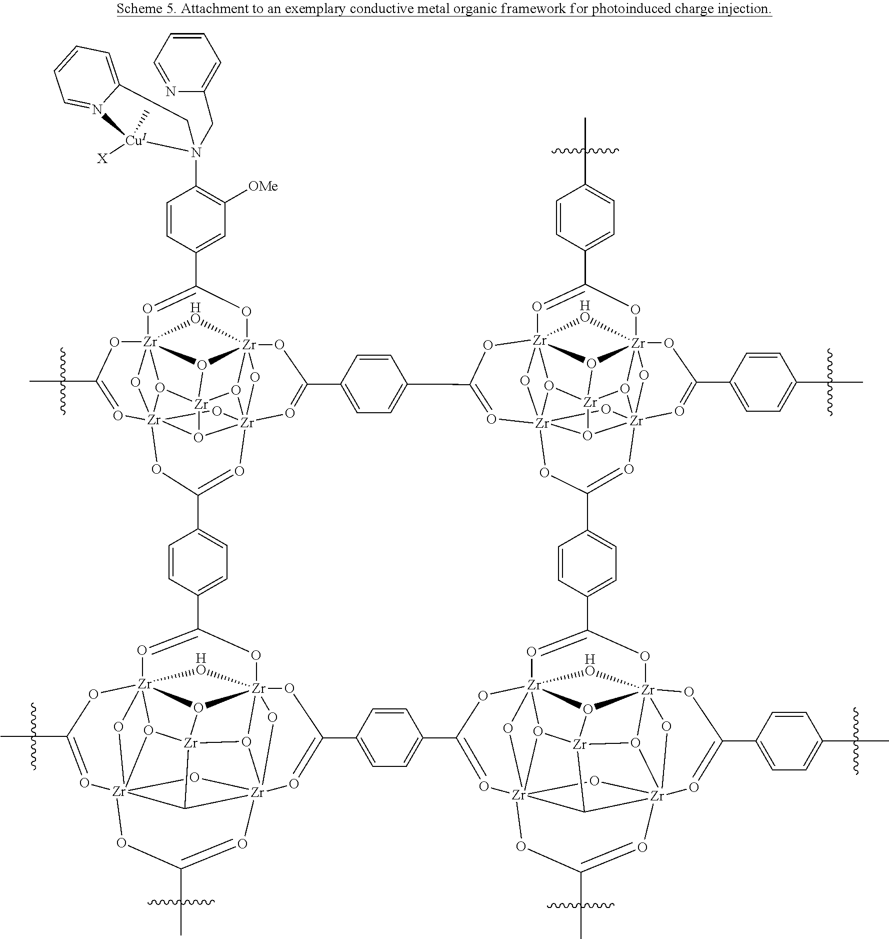

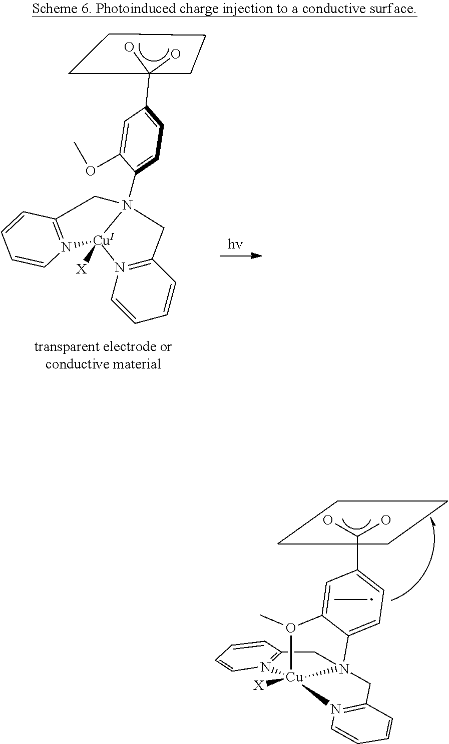

### Example 5. Cyclic Voltammograms for Cu^{I} and Cu^{II} Complexes with Dpa^{H} and Dpa^{OMe}

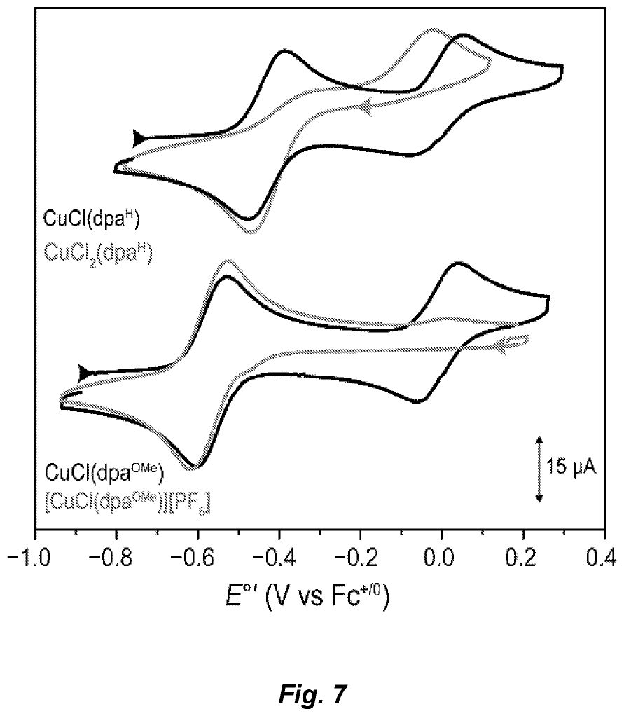

In comparison to the complexes of Formula I, the Cu^{I} complexes that lack an electron withdrawing group on the ligand (Chart 1) and the corresponding Cu^{II} complexes are not photoactive. The cyclic voltammograms (CVs) in FIG. 7 illustrate that the Cu(I) complexes are conformationally fluxional and exhibit two Cu-based redox events, one for each conformation. They also reveal that for the Cu(II) complexes, when no —OMe locking group is present the Cu(II/I) couple becomes irreversible, with oxidative and reductive features split by ˜0.5 V. In contrast, when the —OMe locking group is present, one reversible couple is observed, in the tetragonal conformation.

### Example 6. Preparation and Characterization of Additional Ligands and Complexes

For each of these ligands (L) represented in Chart 2, we have also prepared and characterized the corresponding Cu(I) and Cu(II) complexes formulated as CuCl(L) and [CuCl(L)]+, respectively. Ligand syntheses and metalation reactions are described below.

Chart 2. Ligands with Varying Substitutions.

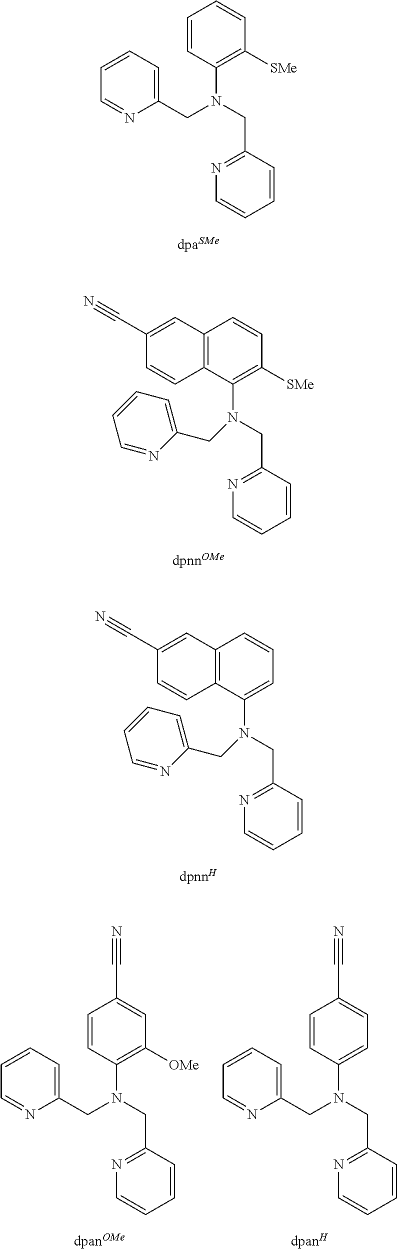 [SMILES: CSc1ccccc1N(Cc1ccccn1)Cc1ccccn1 ; CSc1ccc2cc(C#N)ccc2c1N(Cc1ccccn1)Cc1ccccn1 ; N#Cc1ccc2c(N(Cc3ccccn3)Cc3ccccn3)cccc2c1 ; COc1cc(C#N)ccc1N(Cc1ccccn1)Cc1ccccn1 ; N#Cc1ccc(N(Cc2ccccn2)Cc2ccccn2)cc1]

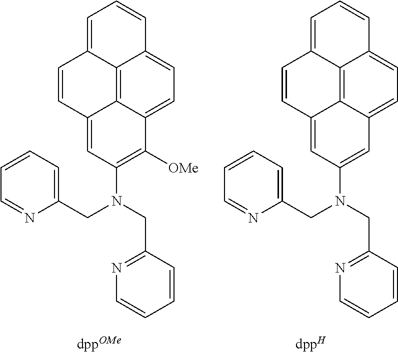 [SMILES: COc1c(N(Cc2ccccn2)Cc2ccccn2)cc2ccc3cccc4ccc1c2c34 ; c1ccc(CN(Cc2ccccn2)c2cc3ccc4cccc5ccc(c2)c3c45)nc1]

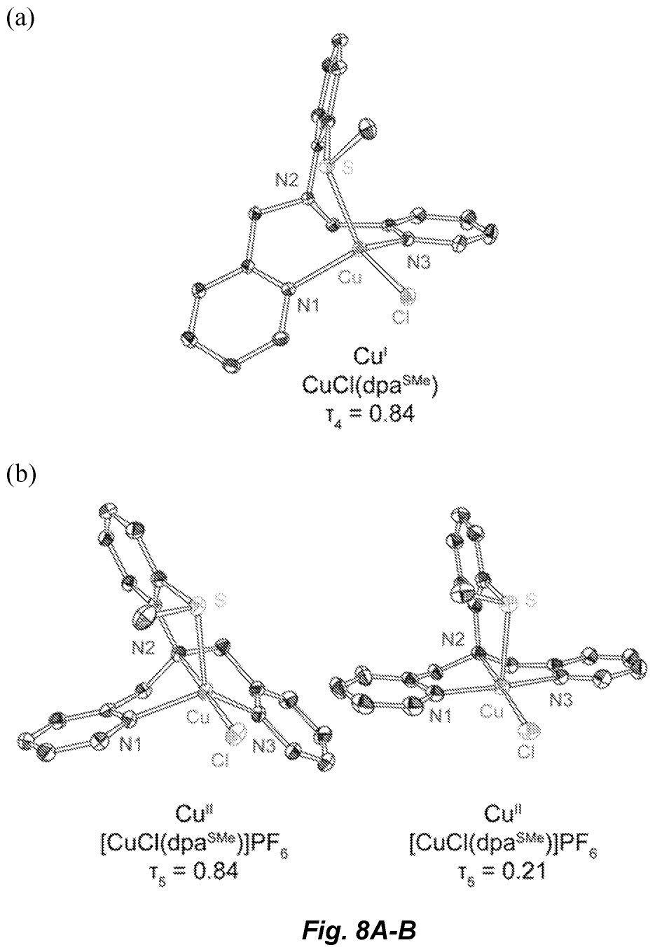

We found that [Cu^{II}Cl(dpa^{SMe})⁺ exhibits dynamicity in this complex, providing a mixture of trigonal bipyramidal and square pyramidal coordination geometries, while the Cu(I) complexes exist in a tetrahedral coordination chemistry. FIG. 8 shows the solid-state X-ray diffraction structures obtained for these complexes. Variable temperature EPR studies support the dynamic interconversion of Cu(II) species in solution.

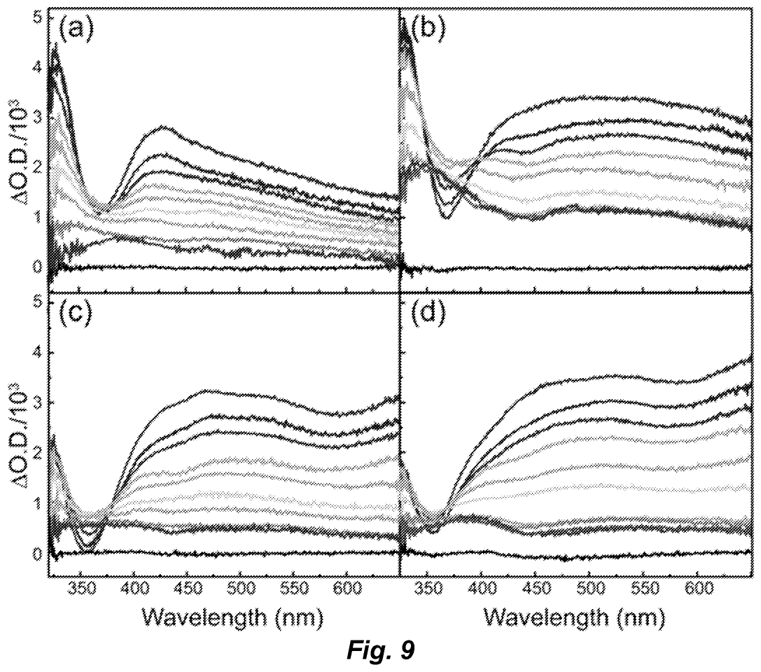

To confirm that the most intense excited state absorbance (ESA) features arises from the presence of the acyl substituent in our dpaa^{R} ligands, we prepared the ligands presented in Chart 2, in which the —Ac substituent of dpaa^{R} is replaced with a —CN substituent to make dpan^{R}. TA spectra collected in dimethylformamide (DMA) are provided in FIG. 9. Here we do not observe the predominance of ligand-centered transitions. This result suggests that we have successfully funneled excited state energy more directly toward our desired TICT* and CS states. Here the TA spectrum of CuCl(dpan^{OMe}) is distinct from that of dpan^{OMe}, and the kinetics of ESA decay are also distinct. Specifically, we observe a longer-lived component in the presence of Cu that is absence in the spectrum of the ligand alone. We hypothesize that this long-lived component may arise from the presence of a CS state.

From time-dependent density-functional theory (TD-DFT) calculations we discovered that formation of a metal-to-TICT* transition may be occurring with CuCl(dpan^{OMe}). Ultimately, whether we drive CS by exciting the ligand and getting subsequent charge transfer from the metal, or by directly exciting from metal-to-ligand, may be of little consequence to our goals. However, the nature of these transitions may affect the wavelengths and intensities with and at which they occur.

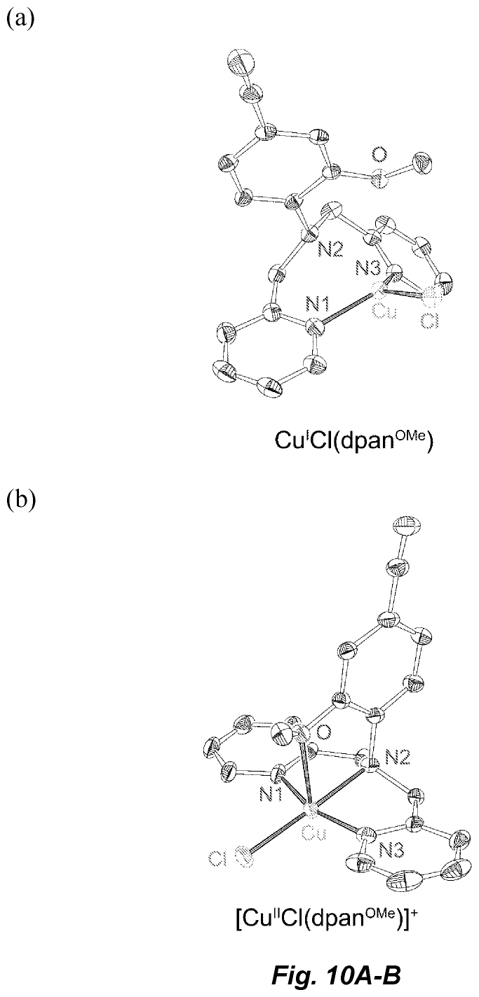

Table 5 lists key bond lengths and angles for both experimentally determined and geometry optimized structures of CuCl(dpan^{OMe}) and [CuCl(dpan^{OMe})]PF₆. The experimentally determined structures, along with the numbering system employed in Table 5, are shown in FIG. 10. Table 1 highlights the large-scale changes observed in both the Cu—N₂ and Cu—O bond lengths/distances, and in the Cl—Cu—N₂ angle on going from Cu(I) to Cu(II). The oxidation state-dependent structural changes are clear: both the Cu—O and Cu—N₂ distances contract by ˜0.5 Å on going from Cu(I) to Cu(II), and the complex rearranges from trigonal to square pyramidal. Thus, we see that performing geometry optimization on the lowest energy excited states of our Cu(I) complexes produce structures that exhibit the clear markings of a Cu(II) state (Table 1, last entry). These computational results support that we have created the desired CS state, which would have Cu(II) character.

TABLE 5
Key Bond Lengths and Angles in Experimental and Geometry Optimized Structures.

| Structure | Cu—N2 (Å) | Cu—O (Å) | Cl—Cu—N2 (°) |
|---|---|---|---|
| Ground state Cu(I), experimental | 2.527 | 2.971 | 130.3 |
| Ground state Cu(I), geometry optimized | 2.554 | 2.961 | 139.2 |
| Ground state Cu(II), experimental | 2.068 | 2.416 | 177.2 |
| Ground state Cu(II), geometry optimized | 2.090 | 2.312 | 171.3 |
| Excited state, geometry optimized | 2.153 | 2.541 | 170.6 |

Also, to push the absorption cross-section of these complexes toward the visible range, we prepared and characterized (by XRD, electrochemistry, UV-vis, emission, IR, NMR, and EPR spectroscopies) the Cu(I) and Cu(II) complexes with the dpnn^{R} ligands shown in Chart 2. Extending the π-system of the ligand did result in an observed redshift of the absorption spectrum.

### Synthesis.

dpa^{SMe}. 2-(methylthio)aniline (1.130 g, 8.12 mmol) was added to a round-bottom flask equipped with stirbar under N₂ and dissolved in DCM (74 mL, 0.11 M). 2-pyridinecarboxaldehyde (2.610 g, 24.4 mmol) was added to the solution and the reaction mixture was allowed to stir for 30 minutes. STAB (5.160 g, 24.4 mmol) was added portionwise and the reaction mixture further permitted to stir overnight. The reaction was extracted with saturated Na₂CO₃ (aq), the aqueous layer rinsed with DCM, the organics subsequently combined and dried over Na₂SO₄, and the organics concentrated under reduced pressure to afford a bright yellow oil. This oil solidified on standing under vacuum overnight to yield a crystalline solid, which was rinsed with 7:3 hexanes/EtOAc and filtered to afford the product as light-yellow crystals (0.794 g, 30%). ¹H NMR (CD₂Cl₂, 600 MHz): 8.45 (dt, J₁=4.72, J₂=1.04 Hz, 2H), 7.63 (d, J=7.74 Hz, 2H), 7.60 (td, J₁=7.23 Hz, J₂=1.66 Hz, 2H), 7.15-7.04 (m, 5H), 6.96 (td, J₁=7.59 Hz, J₂=1.43 Hz, 1H), 4.33 (s, 4H), 2.46 (s, 3H). ¹³C NMR (CD₂Cl₂, 151 MHz): 159.4, 149.2, 147.2, 136.7, 136.6, 125.2, 124.6, 124.3, 122.8, 122.7, 122.3, 59.6, 14.4. HRMS: ESI Positive ion mode m/z [M+1] Calculated. 322.1378 Observed. 322.1380.

[CuCl(dpa^{SMe})]PF₆. dpa^{SMe} (0.100 g, 0.31 mmol) was dissolved in minimal MeOH (2 mL) in a vial equipped with stirbar under air. In a separate vial, CuCl₂(H₂O)₂ (0.058 g, 0.34 mmol) was dissolved in water (1 mL) and the resulting solution added dropwise to the stirring dpaSMe solution. After 15 minutes, NH₄PF₆ (0.203 g, 1.24 mmol) was added and the resulting precipitate was allowed to stir and filtered after 15 minutes. The precipitate was rinsed with water and allowed to dry to afford the product as a cyan solid (0.127 g, 72%). Crystals suitable for XRD could be grown at −20° C. by vapor diffusion of pentane into a solution of complex in DCM or by layer diffusion of a solution of complex into water. EPR (DMF/toluene 1:1, 77K) g_{⊥}: 2.06, g_{∥}: 2.25, A_{∥}167×10⁻⁴ cm⁻¹. HRMS: ESI Positive ion mode m/z [M+] calcd. for: 419.0287, found: 419.0284. Anal. calcd (found) for C₁₉H₁₉CuClN₃SPF₆ (%): C, 40.36 (40.51); H, 3.39 (3.30); N, 7.43 (7.43).

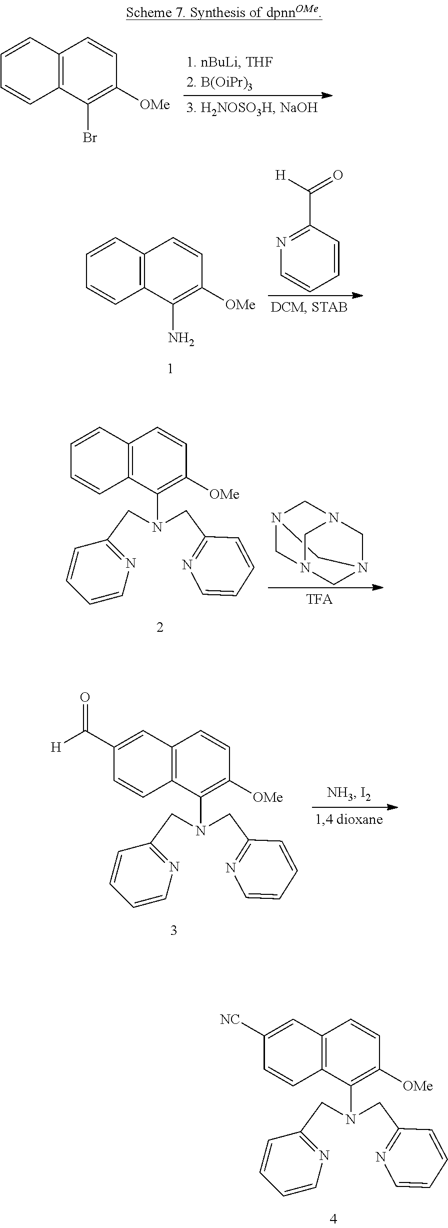

2-methoxynaphthalen-1-amine (1). To an oven dried round bottom flask equipped with a stir bar was added 1-bromo-2-methoxynaphthalene (4 g, 16.9 mmol). The flask was placed under nitrogen atmosphere. THF (85 mL) was transferred to the flask by cannula. The flask was cooled to −78° C. and then n-butyllithium (11.1 mL, 1.6 M in hexanes) was added dropwise to the stirring solution. After 30 minutes, triisopropylborate was added dropwise and then the flask was warmed to room temperature. After 30 minutes, 1 M NaOH (85 mL) and hydroxylamine-O-sulfonic acid (2.9 g, 25.4 mmol) were added. The solution was stirred for 16 hours at room temperature and then diluted with water. The organic layer was separated from the aqueous layer and then the aqueous layer was extracted with ethyl acetate. The organic fractions were combined and evaporated. Silica gel flash column chromatography in 9:1 to 4:1 hexanes to ethyl acetate provided the title compound as a yellow oil in 70% yield. ¹H-NMR (500 MHz, CDCl₃) δ 7.81 (d, J=7.9 Hz, 2H), 7.47 (t, J=8.2 Hz, 1H), 7.38 (m, 2H), 7.28 (d, J=8.8 Hz, 1H). ¹³C NMR (126 MHz, CDCl₃) δ 142.7, 129.7, 129.6, 128.5, 125.1, 124.1, 123.7, 120.4, 118.6, 113.7, 56.8.

2-methoxy-N,N-bis(pyridin-2-ylmethyl)naphthalen-1-amine (2). To a round bottom flask equipped with a stir bar was added (1) (1.63 g, 9.4 mmol) dissolved in DCM (30 mL). To the stirring solution was added picolinealdehyde (1.8 mL, 18.8 mmol). The solution was stirred 30 minutes at room temperature before adding sodium triacetoxyborohydride (4 g, 18.8 mmol). After 2 hours was added picolinealdehyde (2.7 mL, 28.2 mmol). The solution was stirred an additional 30 minutes before adding sodium triacetoxyborohydride (6 g, 28.2 mmol). After stirring 16 hours at room temperature, the reaction was quenched by the addition of water and then extracted with DCM. The organic phase was evaporated. Flash column chromatography was performed on basic alumina Brockmann Grade II in 4:1 hexanes to ethyl acetate. The product was further purified by recrystallization from hexanes/ethyl acetate to provide the title compound as a white solid in 78% yield. ¹H NMR (500 MHz, CDCl₃) δ 8.52 (d, J=8.6 Hz, 1H), 8.46 (m, 2H), 7.71 (d, J=8.3 Hz, 1H), 7.66 (d, J=8.9 Hz, 1H), 7.51 (td, J=7.6, 1.8 Hz, 2H), 7.44 (m, 3H), 7.29 (ddd, J=8.1, 6.7, 1.2 Hz, 1H), 7.21 (d, J=9.0 Hz, 1H), 7.03 (ddd, J=7.3, 4.9, 1.3 Hz, 2H), 4.61 (d, 14.4 Hz, 2H), 4.50 (d, 14.4 Hz, 2H), 3.84 (s, 3H). ¹³C NMR (126 MHz, CDCl₃) δ 160.1, 155.2, 149.0, 136.1, 133.5, 132.5, 129.6, 127.8, 127.2, 126.3, 123.9, 123.6, 123.3, 121.7, 114.2, 77.4, 60.2, 56.0. HRMS (TOF, ESI+) m/z calculated for C₂₃H₂₂N₃O [M+H]⁺: 356.1763, found 356.1765.

5-(bis(pyridin-2-ylmethyl)amino)-6-methoxy-2-naphthaldehyde (3). To a round bottom flask equipped with a stir bar was added (2) (1.23 g, 3.5 mmol) and urotropin (4.9 g, 35 mmol) as solids. Trifluoroacetic acid (12 mL) was added to the flask and the solution was heated to reflux for 36 hours. The solution was cooled to room temperature and then 10% NaOH was added until the pH was above 8. The aqueous layer was extracted with DCM and then the organic phase was washed with saturated sodium bicarbonate and evaporated. The product was recrystallized from hexanes/ethyl acetate to give the title compound as a yellow solid in 60% yield. ¹H NMR (500 MHz, CD₂Cl₂) δ 9.95 (s, 1H), 8.47 (d, J=8.6 Hz, 1H), 8.34 (ddd, J=4.8, 1.8, 0.9 Hz, 2H), 8.10 (d, J=1.7 Hz, 1H), 7.76 (d, J=9.0 Hz, 1H), 7.71 (dd, J=8.9, 1.7 Hz, 1H), 7.44 (td, J=7.6, 1.8 Hz, 3H), 7.30 (dt, J=7.8, 1.1 Hz, 3H), 7.25 (d, J=9.0 Hz, 1H), 6.97 (ddd, J=7.4, 4.8, 1.2 Hz, 3H), 4.46 (d, J=14.2 Hz, 3H), 4.34 (d, J=14.0 Hz, 2H), 3.81 (s, 3H). ¹³C NMR (126 MHz, CD₂Cl₂) δ 192.3, 160.0, 158.5, 149.2, 137.2, 136.3, 135.0, 133.0, 132.7, 129.4, 128.6, 125.5, 123.6, 122.9, 122.1, 115.2, 60.2, 56.2.

5-(bis(pyridin-2-ylmethyl)amino)-6-methoxy-2-naphthonitrile (4). To a round bottom flask equipped with a stir bar was added (3) (0.78 g, 2.03 mmol) as a solid. (3) was dissolved in a minimal amount of 1,4 dioxane. To this solution was added 28% ammonia in water (20 mL) and then iodine (1 g, 4 mmol). The solution was stirred for 24 hours at room temperature and then was quenched with saturated sodium bisulfite. The 1,4 dioxane was evaporated and then the aqueous layer was extracted with DCM. The organic phase was evaporated, and the resulting solid was recrystallized from hexanes/ethyl acetate to give the title compound as a yellow solid in 43% yield. ¹H NMR (600 MHz, CDCl₃) δ 8.55 (d, J=8.8 Hz, 1H), 8.47-8.42 (m, 2H), 8.05 (d, J=1.7 Hz, 1H), 7.70 (d, J=9.0 Hz, 1H), 7.52 (td, J=7.6, 1.8 Hz, 2H), 7.47 (dd, J=8.8, 1.6 Hz, 1H), 7.36 (d, J=7.7 Hz, 2H), 7.31 (d, J=9.0 Hz, 1H), 7.05 (dd, J=7.5, 4.9 Hz, 2H), 4.57 (d, J=14.1 Hz, 2H), 4.44 (d, J=14.1 Hz, 2H), 3.90 (s, 3H). ¹³C NMR (151 MHz, CDCl₃) δ 159.2, 157.8, 149.0, 136.1, 135.2, 134.0, 132.4, 128.0, 127.9, 126.4, 125.2, 123.3, 121.9, 119.8, 115.3, 106.6, 60.0, 55.9. HRMS (TOF, ESI+) m/z calculated for C₂₄H₂₁N₄O [M+H]⁺: 381.1717, found 381.1715.

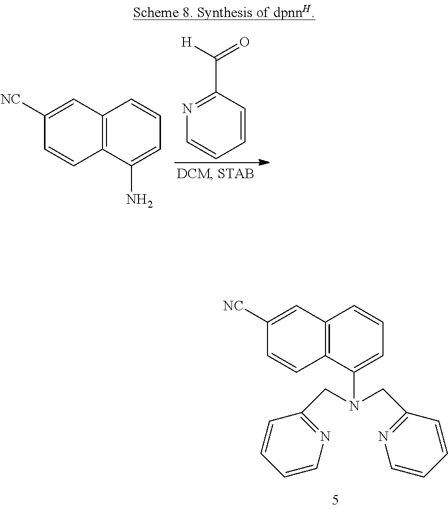

5-(bis(pyridin-2-ylmethyl)amino)-2-naphthonitrile (5). To a round bottom flask equipped with a stir bar was added 5-amino-2-naphthonitrile (0.25 g, 1.5 mmol) dissolved in DCM (10 mL). To the stirring solution was added picolinealdehyde (0.29 mL, 3 mmol). The solution was stirred 30 minutes at room temperature before adding sodium triacetoxyborohydride (0.6 g, 3 mmol). After 2 hours was added picolinealdehyde (0.45 mL, 4.5 mmol). The solution was stirred an additional 30 minutes before adding sodium triacetoxyborohydride (0.9 g, 4.5 mmol). After stirring 24 hours at room temperature, the reaction was quenched by the addition of water and then was extracted with DCM. The organic phase was evaporated. Flash column chromatography was performed on basic alumina Brockmann Grade II in 4:1 hexanes to ethyl acetate. The product was further purified by recrystallization from hexanes/ethyl acetate to provide the title compound as a light-yellow solid in 41% yield.

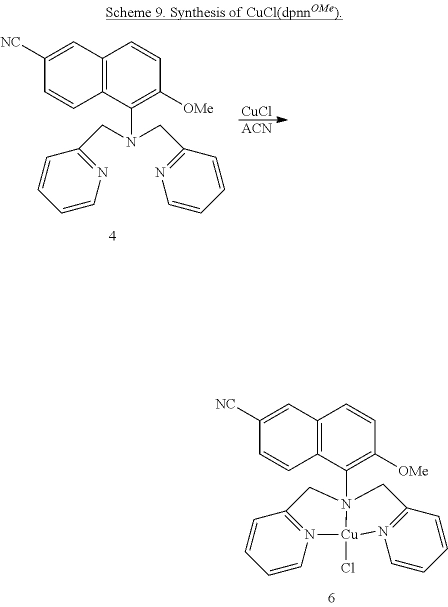

Cu(5-(bis(pyridin-2-ylmethyl)amino)-6-methoxy-2-naphthonitrile)Cl (6). In a glovebox, 4 was dissolved in a minimal amount of acetonitrile (ACN) and then CuCl was added as a solid. The reaction was stirred in the glovebox overnight and then the reaction was filtered through Celite. The ACN was then removed under reduced pressure. The resulting oil was dissolved in a small amount of DCM and then pentane was added to cause precipitation of the complex. The solvent was decanted and then this process was repeated three times to give the title complex in 50% yield. ¹H NMR (600 MHz, CD₂Cl₂) δ 9.02 (bs, 2H), 8.70 (bs, 1H), 8.16 (bs, 1H), 7.85 (d, J=8.5 Hz, 1H), 7.73 (bs, 2H), 7.56 (d, J=7.3 Hz, 1H), 7.37 (m, 2H), 7.09 (bs, 2H), 4.64 (bs, 2H), 4.46 (bs, J=14.1 Hz, 2H), 3.85 (bs, 3H).

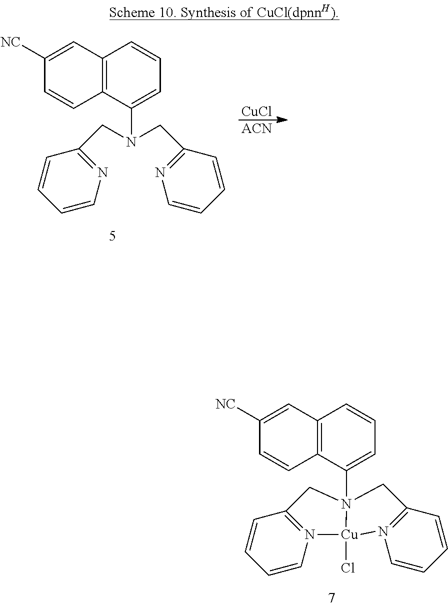

Cu(5-(bis(pyridin-2-ylmethyl)amino)2-naphthonitrile)Cl (7). In a glovebox, 5 was dissolved in a minimal amount of acetonitrile (ACN) and then CuCl was added as a solid. Almost immediately after adding the CuCl, a yellow precipitate formed. The reaction was stirred in the glovebox overnight. The ACN was then removed under reduced pressure. The resulting powder was dissolved in a small amount of DCM and then pentane was added to cause precipitation of the complex. The solvent was decanted to give the title complex in 99% yield.

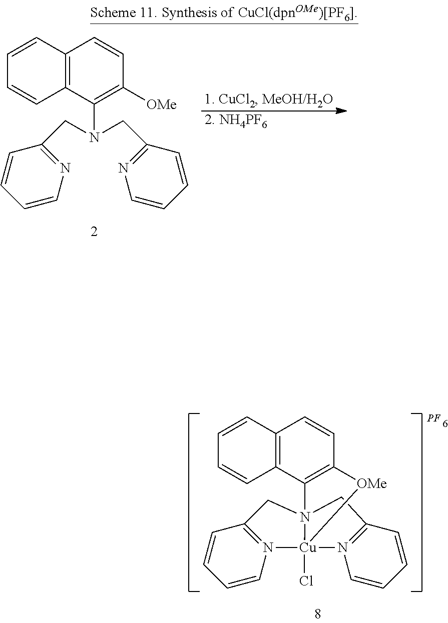

Cu(2-methoxy-N,N-bis(pyridin-2-ylmethyl)naphthalen-1-amine)Cl[PF₆](6). To a round bottom flask was added 2, which was then dissolved in MeOH. CuCl₂ dissolved in H₂O was then added. The solution was stirred for one hour at room temperature and then NH₄PF₆ was added as a solid. A blue precipitate immediately formed and then the reaction was filtered and the blue solid was washed with hexanes and DCM. The blue solid was collected and dried to give the title compound in 90% yield.

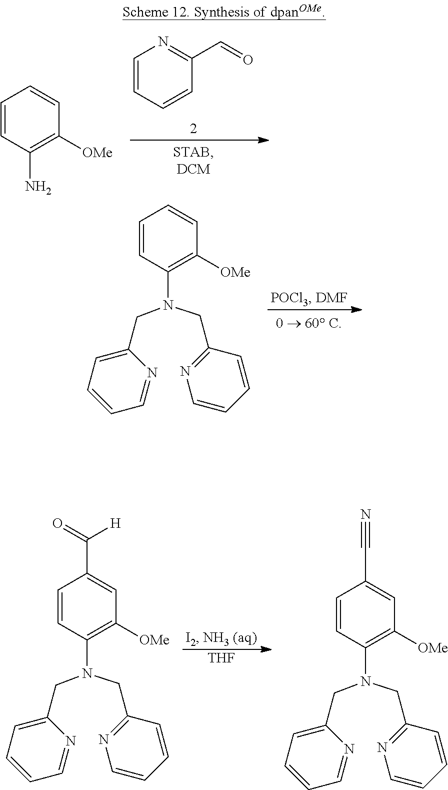 [SMILES: COc1ccccc1N ; O=Cc1ccccn1 ; COc1ccccc1N(Cc1ccccn1)Cc1ccccn1 ; COc1cc(C=O)ccc1N(Cc1ccccn1)Cc1ccccn1 ; COc1cc(C#N)ccc1N(Cc1ccccn1)Cc1ccccn1 ; "THF"]

2-Methoxy-N,N-bis(pyridine-2-ylmethyl)aniline. To a RB equipped with N₂ and stir was added o-anisidine (1.00 g, 8.12 mmol) and DCM (150 mL). 2-pyridine carboxaldehyde (2.60 g, 24.2 mmol) was then added and the solution was allowed to stir for 30 minutes under N₂. After 30 min, sodium triacetoxyborohydride (5.20 g, 24.5 mmol) was added portion wise, and the reaction was allowed to proceed for an additional 16 h. The crude reaction mixture was extracted with sat. Na₂CO₃, dried over Na₂SO₄, and the solvent removed in vacuo. The crude product was subjected to column chromatography on basic alumina with 7:3 hexanes:EtOAc to provide the product as a beige solid (0.68 g, 60%). ¹H-NMR (500 MHz, CDCl₃): δ 3.84 (s, 3H), 4.52 (s, 4H), 6.74 (t, 1H), 6.88 (m, 3H), 7.08 (t, 2H), 7.48 (d, 2H), 7.56 (t, 2H), 8.48 (d, 2H). ESI-MS positive ion mode m/z [M⁺] calcd: 306.4, found: 306.1.

4-(bis(pyridine-2-ylmethyl)amino)-3-methoxybenzaldehyde. To a RB equipped with N₂ and stir was added DMF (5 mL) chilled to 0° C., followed by dropwise addition of POCl₃ (5 mL). The resulting solution was allowed to stir at 0° C. for 30 min before 2-methoxy-N,N-bis(pyridine-2-ylmethyl)aniline (0.30 g, 0.98 mmol) in DMF (2.5 mL) was added dropwise. The reaction was warmed to room temperature then warmed to 60° C. for 18 h. The reaction mixture was cooled to room temperature then poured into an ice water solution (150 mL) with vigorous stirring. Sat. Na₂CO₃ was slowly added to neutralize before extraction with DCM. The resulting organic layer was dried over Na₂SO₄ and solvent was removed under reduced pressure. The resulting crude oil was subjected to column chromatography (2:1 hexanes:EtOAc on basic alumina) to yield the product as a light-yellow oil (0.06 g, 20%). ¹H-NMR (400 MHz, CD₂Cl₂): δ 3.77 (s, 3H), 4.68 (s, 4H), 6.85 (d, 1H), 7.11 (t, 2H), 7.22 (t, 2H), 7.35 (d, 2H), 7.37 (s, 1H), 7.58 (t, 2H), 8.49 (d, 2H), 9.72 (s, 1H). ESI-MS positive ion mode m/z [M⁺] calcd: 334.4, found: 334.2.

4-(bis(pyridin-2-ylmethyl)amino)-3-methoxybenzonitrile (dpan^{OMe}). To a round bottom equipped with stir, iodine (0.21 g, 0.83 mmol) in THF (1 mL) was added to a solution of 4-(bis(pyridine-2-ylmethyl)amino)-3-methoxybenzaldehyde (0.21 g, 0.63 mmol) in 28% aq. NH₃ (15 mL, 28% solution). The reaction was allowed to run for 1 h before recharging with iodine (0.16 g, 0.63 mmol) and aq. NH₃ (7 mL 28% v/v) and running for an additional 2 h. The reaction was quenched with 10 mL of 5% aq. Na₂S₂O₃ and extracted with DCM. The organic layer was dried over Na₂SO₄ and DCM was removed under reduced pressure to yield the product as a golden yellow solid (0.18 g, 89%). ¹H-NMR (500 MHz, CDCl₃): δ 3.78 (s, 3H), 4.65 (s, 4H), 6.84 (d, 1H), 7.03 (d, 1H), 7.07 (dd, 1H), 7.14 (t, 2H), 7.37 (d, 2H), 7.62 (t, 2H), 8.53 (d, 2H). ESI-MS positive ion mode m/z [M⁺] calcd: 331.4, found: 331.3.

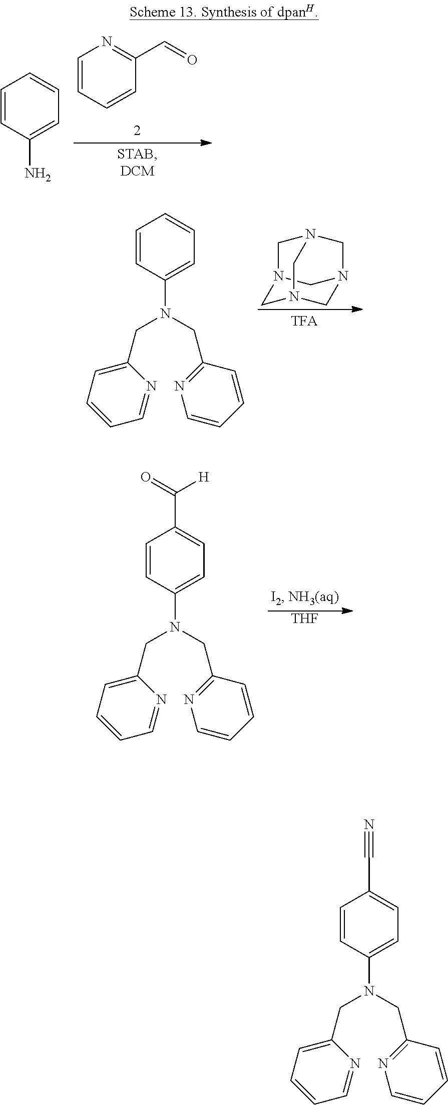

N,N-bis(pyridine-2-ylmethyl)aniline. To a RB flask equipped with N₂ and stir was added aniline (0.93 g, 9.99 mmol) and DCM (130 mL). 2-pyridine carboxaldehyde (4.73 g, 44.2 mmol) was then added and the solution was allowed to stir for 30 min at 40° C. under N₂. The solution was cooled to RT and STAB (8.40 g, 39.8 mmol) was added dropwise. The reaction was allowed to proceed for an additional 48 h. The crude reaction mixture was then extracted with sat. Na₂CO₃, dried over Na₂SO₄, and concentrated in vacuo. The crude product was subjected to column chromatography on basic alumina with 2:1 hexanes:EtOAc to provide the product as an off-white solid (1.50 g, 54%). ¹H-NMR (500 MHz, CDCl₃): δ 4.86 (s, 4H), 6.74 (m, 3H), 7.20 (m, 4H), 7.30 (d, 2H), 7.65 (td, 2H), 8.63 (d, 2H). ESI-MS positive ion mode m/z [M⁺] calcd: 276.1, found: 276.2.

4-(bis(pyridine-2-ylmethyl)amino)-benzaldehyde. To a RB flask equipped with N₂ and stir was added N,N-bis(pyridine-2-ylmethyl)aniline (0.35 g, 1.27 mmol) in trifluoroacetic acid (10 mL) followed by addition of hexamethylenetetramine (1.80 g, 12.8 mmol). The reaction solution was refluxed for 36 h then neutralized with NaHCO₃ before extraction into DCM. The organic layer was dried over Na₂SO₄ and concentrated in vacuo. The crude product was subjected to column chromatography (1:1 hexames:EtOAc on basic alumina) to yield the product as a yellow solid (0.31 g, 79%). ¹H-NMR (500 MHz, CDCl₃): δ 4.91 (s, 4H), 6.79 (d, 2H), 7.21 (m, 4H), 7.66 (m, 4H), 8.61 (dd, 2H), 9.73 (s, 1H). ESI-MS positive ion mode m/z [M⁺] calcd: 304.4, found: 304.1.

4-(bis(pyridine-2-ylmethyl)amino)-benzonitrile. To a RB flask equipped with N₂ and stir was added 4-(bis(pyridine-2-ylmethyl)amino)-benzaldehyde (0.30 g, 1.00 mmol) and aq. NH₃ (28% v/v, 15 mL) and THF (1.5 mL). A solution of I₂ (0.33 g, 1.31 mmol) in THF (1 mL) was then added and the reaction allowed to stir for 1.5 h. The reaction was then recharged with an additional 1 eq. I₂ in THF (0.5 mL) and NH₃ (28% aq., 10 mL). After 2 h, the reaction was quenched with 10 mL of 5% aq. Na₂S₂O₃ and extracted with DCM. The organic layer was dried over Na₂SO₄, and DCM was removed under reduced pressure to yield the product as a yellow oil (0.30 g, quant.). ¹H-NMR (600 MHz, CDCl₃): δ 4.92 (s, 4H), 6.75 (d, 2H), 7.29 (m, 4H), 7.47 (d, 2H), 7.75 (td, 2H), 8.56 (dt, 2H). ESI-MS positive ion mode m/z [M⁺] calcd: 301.3, found: 301.0.

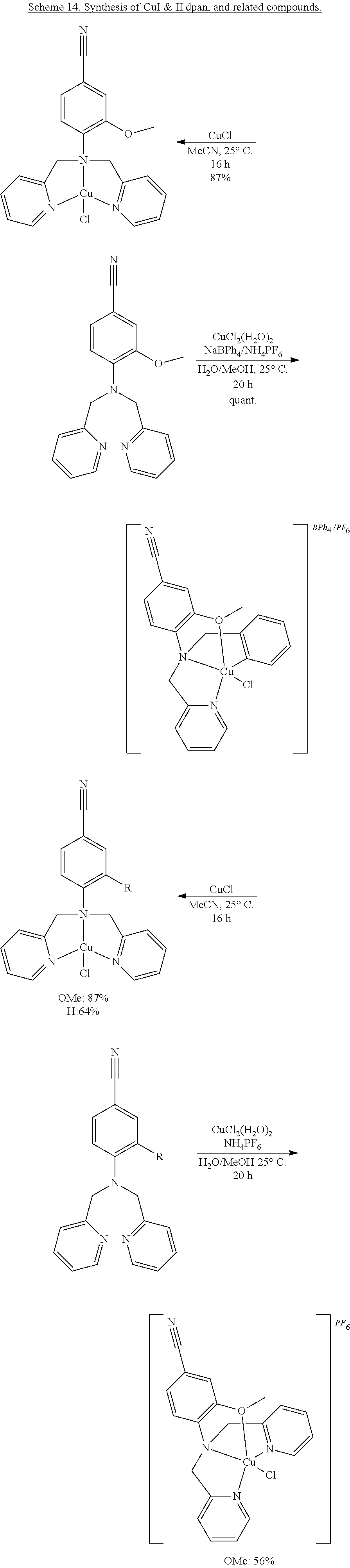

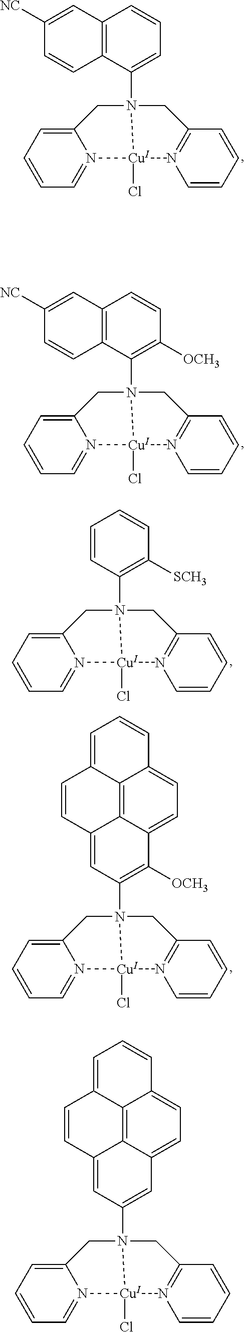

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A metal complex of Formula I: 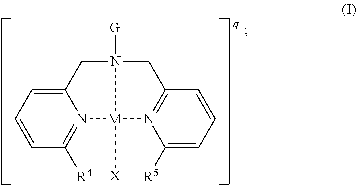
wherein [symbol]
is a dative or a covalent bond;
G is: 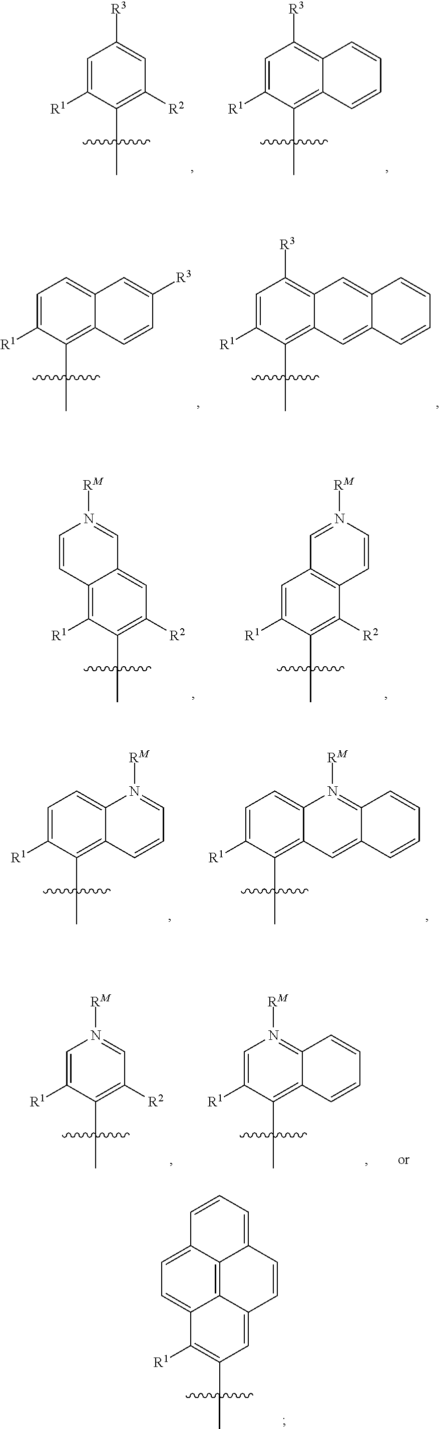
M is a period 4 transition metal or an ion thereof;
R¹ is —O(C₁-C₆)alkyl, —S(C₁-C₆)alkyl, —N[(C₁-C₆)alkyl]₂, —P[(C₁-C₆)alkyl]₂, or —B[(C₁-C₆)alkyl]₂, wherein the heteroatom moiety of R¹ is optionally bonded to M;
R² is H, —O(C₁-C₆)alkyl, —S(C₁-C₆)alkyl, —N[(C₁-C₆)alkyl]₂, —P[(C₁-C₆)alkyl]₂, or —B[(C₁-C₆)alkyl]₂;
R³ is —C(═O)(C₁-C₆)alkyl, —C(═O)OH, —CN, —S(═O)₂(C₁-C₆)alkyl, —S(═O)₂O(C₁-C₆)alkyl, or —C(═O)Ph wherein Ph is optionally substituted;
R⁴ and R⁵ are H, or CH₂ bonded to X;
R is a metal center or complex thereof, O, or a lone pair electrons;
X is halo, or a nitrogen or oxygen heterocycle, when R⁴ and R are H; or
X, when R⁴ and R⁵ are CH₂, is —N(C₁-C₆)alkyl, —P(C₁-C₆)alkyl, —B[(C₁-C₆)alkyl]_{1 or 2}, —B(halo)_{1 or 2}, O, or S; and
q is absent or a counter ion.

2. The metal complex of claim 1 wherein M is Cu(I), Cu(II), Ni(0), Ni(II), Pd(0), Pd(II), Pt(0), or Pt(II); and R^{M} is optionally a metal center or complex thereof and the metal center comprises an ion of Co, Fe, Os, or Ru.

3. The metal complex of claim 2 wherein M is Cu(I) and q is absent.

4. The metal complex of claim 2 wherein M is Cu(II) and q is a counter ion.

5. The metal complex of claim 1 wherein R¹ is —O(C₁-C₆)alkyl.

6. The metal complex of claim 1 wherein G is:

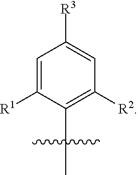

7. The metal complex of claim 6 wherein R³ is —C(═O)(C₁-C₆)alkyl.

8. The metal complex of claim 1 wherein R⁴ and R⁵ are H.

9. The metal complex of claim 1 wherein X is halo.

10. The metal complex of claim 1 wherein Formula I is represented by Formula II:

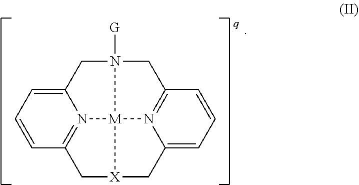

11. The metal complex of claim 10 wherein X is —N(C₁-C₆)alkyl.

12. The metal complex of claim 1 wherein the metal complex is Ia or Ib: 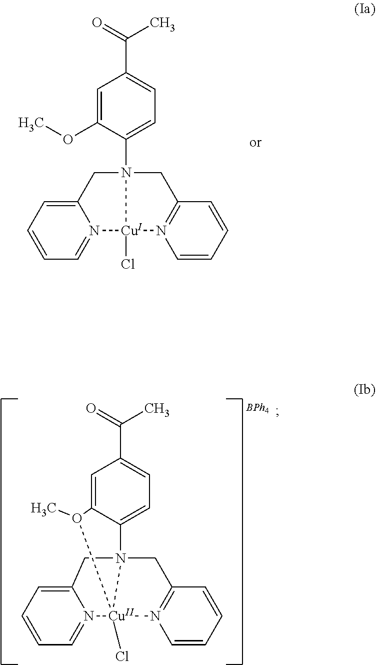
wherein the coordination geometry of Cu(I) of complex Ia is trigonal, or the coordination geometry of Cu(II) of complex Ib is tetragonal.

13. A semiconductor or composition comprising a metal complex of claim 1.

14. A compound of Formula III: 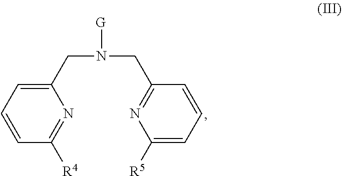
or a salt thereof;
wherein
G is: 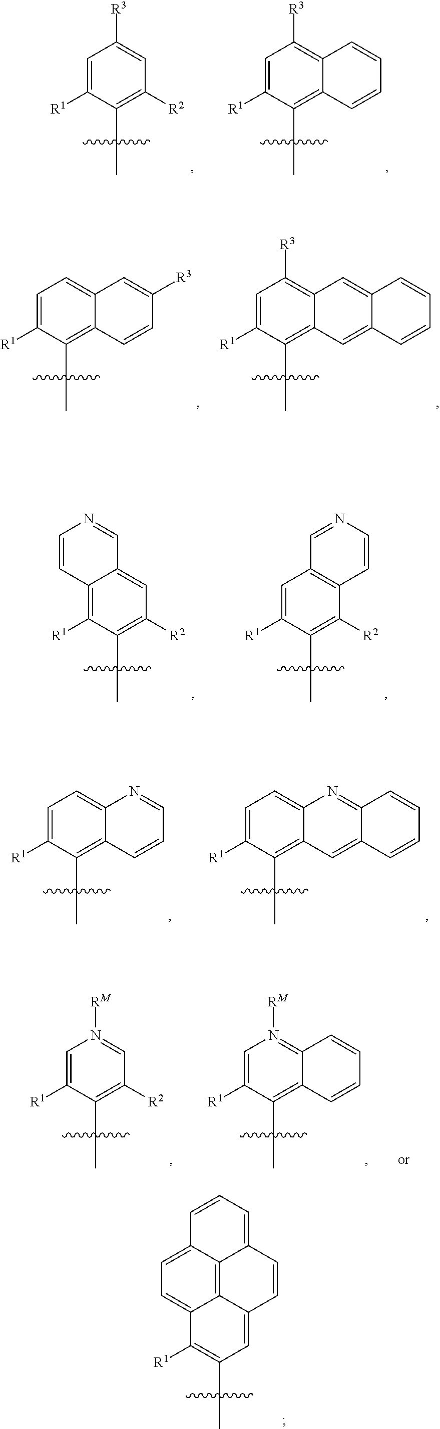
R¹ is —O(C₁-C₆)alkyl, —S(C₁-C₆)alkyl, —N[(C₁-C₆)alkyl]₂, —P[(C₁-C₆)alkyl]₂, or —B[(C₁-C₆)alkyl]₂;
R² is H, —O(C₁-C₆)alkyl, —S(C₁-C₆)alkyl, —N[(C₁-C₆)alkyl]₂, —P[(C₁-C₆)alkyl]₂, or —B[(C₁-C₆)alkyl]₂;
R³ is —C(═O)(C₁-C₆)alkyl, —C(═O)OH, —CN, —S(═O)₂(C₁-C₆)alkyl, or —S(═O)₂O(C₁-C₆)alkyl, or —C(═O)Ph wherein Ph is optionally substituted;
R⁴ and R⁵ are H; or
R⁴ and R⁵ taken together is —CH₂—X—CH₂—; and
X is —N(C₁-C₆)alkyl, —P(C₁-C₆)alkyl, —B[(C₁-C₆)alkyl]_{1 or 2}, —B(halo)_{1 or 2}, O, or S.

15. The compound of claim 14 wherein G is:

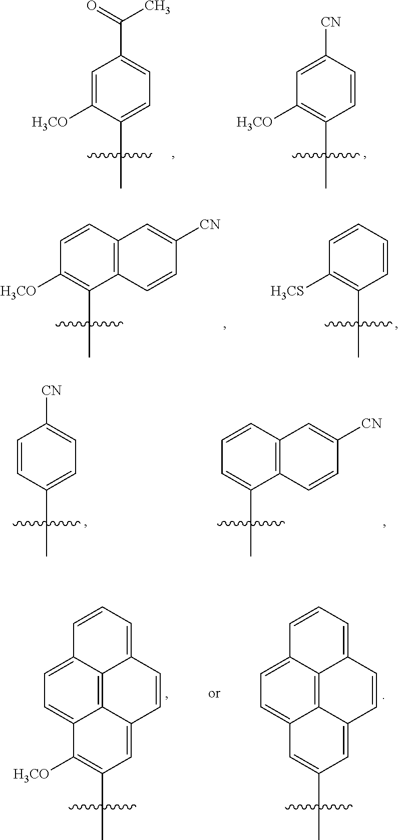

16. The compound of claim 14 wherein the compound is IIIa: 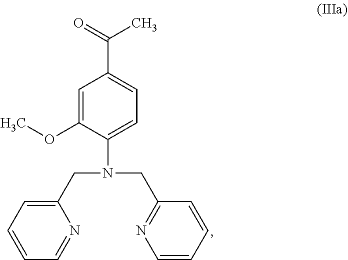 [SMILES: COc1cc(C(C)=O)ccc1N(Cc1ccccn1)Cc1ccccn1]
or a salt thereof.

17. A composition comprising a compound of claim 14 and another substance.

18. A method for stabilizing charge separation in a transition metal coordination complex comprising:
irradiating a transition metal coordination complex at a suitable excitation wavelength to form a stabilized charge separated state in the complex via a twisted intramolecular charge transfer (TICT) process;
wherein the complex comprises:
i) a transition metal at a first oxidation state;
ii) a fluorophore wherein one of its emitting states is comprised of a radical anion localized within the fluorophore;
iii) a metal coordinating heteroatom covalently bonded to the fluorophore; and
iv) an electron withdrawing group (EWG) covalently bonded to the fluorophore wherein the EWG is bonded in a position where it is optimally capable of stabilizing the radical anion; and
v) a multidentate ligand covalently bonded to the fluorophore via a second metal coordinating heteroatom;
wherein the second metal coordinating heteroatom forms a transient radical cation and the fluorophore forms a transient radical anion when the complex is in a TICT state triggered by the irradiation;
the fluorophore has sufficient degrees of freedom to adopt a conformation that allows formation of a dative bond to the transition metal via the metal coordinating heteroatom;
the complex undergoes a conformational rearrangement that stabilizes the charge separated state in the complex; and
the transition metal of the complex in the charge separated state is oxidized to a second oxidation state.

19. The method of claim 18 wherein the transition metal coordination complex is represented by Formula I: 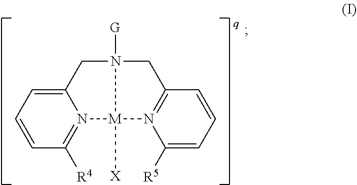
wherein [symbol]
is a dative or a covalent bond;
G is: 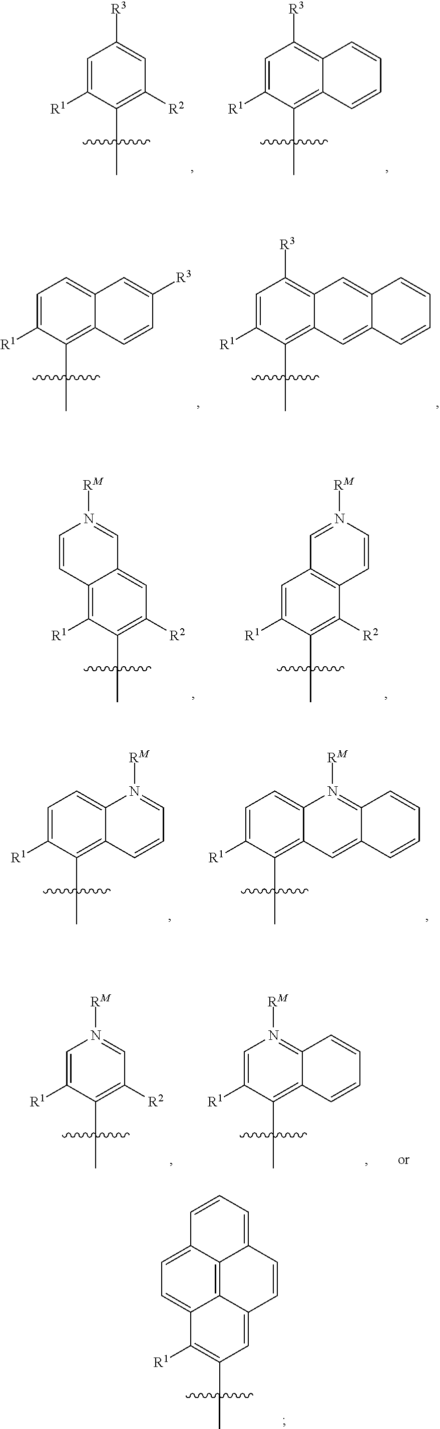
M is a period 4 transition metal or an ion thereof;
R¹ is —O(C₁-C₆)alkyl, —S(C₁-C₆)alkyl, —N[(C₁-C₆)alkyl]₂, —P[(C₁-C₆)alkyl]₂, or —B[(C₁-C₆)alkyl]₂, wherein the heteroatom moiety of R¹ is optionally bonded to M;
R² is H, —O(C₁-C₆)alkyl, —S(C₁-C₆)alkyl, —N[(C₁-C₆)alkyl]₂, —P[(C₁-C₆)alkyl]₂, or —B[(C₁-C₆)alkyl]₂;
R³ is —C(═O)(C₁-C₆)alkyl, —C(═O)OH, —CN, —S(═O)₂(C₁-C₆)alkyl, or —S(═O)₂O(C₁-C₆)alkyl, or —C(═O)Ph wherein Ph is optionally substituted;
R⁴ and R⁵ are H or CH₂ bonded to X;
R^{M} is a metal center or complex thereof, O, or a lone pair electrons;
X is halo, or a nitrogen or oxygen heterocycle, when R⁴ and R are H; or
X, when R⁴ and R⁵ are CH₂, is —N(C₁-C₆)alkyl, —P(C₁-C₆)alkyl, —B[(C₁-C₆)alkyl]_{1 or 2}, —B(halo)_{1 or 2}, O, or S; and
q is absent or a counter ion.

20. The method of claim 18 wherein the transition metal coordination complex is (Ia): [chemical structure]
